(19) United States
(12) Patent Application Publication
(10) Pub. No.: US 2002/0150541 A1
(43) Pub. Date: Oct. 17, 2002

(54) Compositions comprising mixtures of therapeutic proteins and methods of producing the same

(75) Inventors: Allan S. Lau, Pok Fu Lam (HK); Winnie H. Wan, Woodside, CA (US); Laura Browning, Brentwood, CA (US); Natalya Ossina, Albany, CA (US)

Correspondence Address: PERKINS COIE LLP, P.O. BOX 2168, MENLO PARK, CA, 94026, US

(73) Assignee: Gene Trol Biotherapeutics, Inc.

(21) Appl. No.: 10/105,100

(22) Filed: Mar. 21, 2002

Related U.S. Application Data
(63) Continuation-in-part of application No. 09/952,843, filed on Sep. 11, 2001.
Application No. 09/952,843 is a continuation-in-part of application No. 09/660,468, filed on Sep. 12, 2000.

(51) Int. Cl. A61K 38/21; A61K 9/14; A61L 9/04

(52) U.S. Cl. 424/46; 424/85.6; 424/85.7

(57) ABSTRACT

Human cytokine mixtures produced by cytokine regulatory factor-overexpressing cells and methods of production are disclosed. The mixtures are prepared by culturing human cytokine-producing cells under conditions of cytokine regulatory factor overexpression, treating the cells to induce cytokine production, and isolating the mixtures of cytokines produced by the cells. Exemplary compositions include mixtures of human interferon γ in combination with human interferon α and/or human interferon β, and mixtures of human interferon α and human interferon β. Also disclosed are therapeutic uses of the interferon compositions.

This application is a continuation-in-part of U.S. patent application Ser. No. 09/952,843, filed Sep. 11, 2001 which is a continuation-in-part of U.S. patent application Ser. No. 09/660,468, filed Sep. 11, 2000, which is incorporated herein in its entirety by reference.

### FIELD OF THE INVENTION

The present invention relates to therapeutic compositions which comprise selected mixtures of cytokines for use in treating viral infections, cancers, inflammatory disorders, other cytokine-responsive disorders and methods for producing the same by human cell lines. a—

References

Altschul, S. F. et al., J. Mol. Biol. 215:403-410, 1990.

Altschul, S. F. et al., Nucleic Acids Res. 25:3389-3402, 1997.

Ausubel F M et al., Current Protocols in Molecular Biology, John Wiley & Sons, New York, N.Y., 1989.

Barber, G. N. et al., Proc. Natl. Acad. Sci. USA 91, 4278-4282, 1994.

Clemens M J and Bommer U A, Int J Biochem Cell Biol, 31(1):1-23, 1999.

Cohen, J. J., Immunol Today 14(3):126-130,1993.

Der, D., and Lau, A. S., Proc Natl Acad Sci USA, 92:8841-8845, (1995).

Donze, O., et al., Virol 256:322-329, 1999.

Galabru, J., and Hovanessian, A., J. Biol. Chem. 262:15538-15544, 1987.

Hershey, J. W. B., Ann. Rev. Biochem. 60:717-755, 1991.

Hopp et al., Biotechnology 6: 1204-1210, 1988.

Hovanessian, Biochimie 62:775-778, 1980.

Huang et al., Oncogene 14: 405-414, 1997.

Inoue I et al. (Neurol Med Chir (Tokyo) 31 (8):484-489, 1991).

Jagus R. et al., Int J Biochem Cell Biol. 31(1):123-38, 1999.

Koromilas et al., Science 257:1685, 1992.

Kumar, A., et al Proc Natl Acad Sci USA 91:6288-6292,1994.

Larrick, J. W., and Wright, S. C., FASEB J 4:3215-3223, 1990.

Liddil, J. D., et al., Cancer Res 49:2722-2728, 1989.

Meurs, E., et al., Cell 62:379-390, 1990.

Mulligan and Berg, 1980.

Orrenius, S., J Intern Med 237:529-536, 1995.

Patel, R. C. and Sen, G. C. EMBO J., 17, 4379-4390, 1998.

Rubin, B. Y., et al., Cancer Res 48:6006-6010, 1988.

Sambrook J et al., in MOLECULAR CLONING: A LABORATORY MANUAL, Cold Spring Harbor Laboratory Press, Cold Spring Harbor, N.Y., Vol. 2, 1989.

Southern and Berg, J Mol Appl Genet. 1(4):327-41, 1982.

Srivastave, S., et al., J Biol Chem 273:2416-2423, 1998.

Stellar, H., Science 267:1445-1449, 1995.

Sugden et al, Mol. Cell. Biol., 5: 410-413, 1985.

Vaux, D. L., Proc Natl Acad Sci USA 90:786-789, 1993.

Wek R C, Trends Biochem Sci; 19: 491-496, 1994.

Williams B R, Oncogene 18(45):6112-20, 1999.

Yeung, M. C., et al., Proc Natl Acad Sci USA 93:12451-12455, 1996.

Yeung M C, et al., Proc Natl Acad Sci USA 96(21):11860-5, 1999.

### BACKGROUND OF THE INVENTION

Cytokines are useful in treating a number of human pathologies, including cancers, viral infections, and inflammation. Typically, cytokine treatment involves administering a single, isolated and generally recombinant cytokine, such as interferon-alpha (IFN-alpha), interferon-beta (IFN-beta) or tissue necrosis factor (TNF), etc. Although some treatment efficacy has been observed, the extent of improvement is suboptimal and the cytokines are generally administered in combination with one or more additional treatment modalities.

In vivo cytokine-producing cells, such as monocytes, macrophages, B cells, dendritic cells, T_{H}1 and T_{H}2, mast cells, NK cells and bone-marrow stromal cells, typically produce complex mixtures of cytokines. Hence, it is not surprising that a single cytokine, when administered alone, is not optimally effective.

It would therefore be desirable to prepare, for clinical use, a cytokine composition comprising a mixture of cytokines, which is similar to a natural mixture of cytokines, produced in vivo. It would be particularly desirable to produce such a cytokine mixture in a cell culture system that produces and secretes high levels of cytokines.

parent application Ser. No. 09/595,338 and its provisional predecessor application disclose a cell culture method of making high levels of cytokines, by growing cytokine-producing human cells under conditions of cytokine regulatory factor overexpression and cytokine induction. The present application is concerned with cytokine mixtures produced by a related method, to methods of obtaining improved cytokine compositions, and to methods of use of the compositions comprising mixtures of cytokines.

### SUMMARY OF THE INVENTION

One aspect of the invention provides a composition comprising a mixture of human interferon γ and at least one of human interferon α and human interferon β, in a mole ratio of between 1:1 to 1:1000 interferon γ to interferon α or human interferon β. In exemplary embodiments, the composition includes human interferon γ and either or both of interferon α and human interferon β, at a mole ratio of between 1:10 to 1:100 interferon γ to interferon α and/or interferon β. The interferon α may include a mixture of interferon α subtypes. In one embodiment, the composition is formulated in a pharmaceutically injectable solution. In another embodiment the composition of claim 1, which is formulated for dispersion in a particle aerosol, for administration by inhalation. In another embodiment, the composition is formulated in a dry powder form.

Another aspect of the invention is a method for treating a condition in a human subject that is responsive to human interferon α or human interferon β, when administered in a therapeutically effective dose to the subject, comprising administering to the subject, a dose of human interferon α or human interferon β that is below the dose required for therapeutic efficacy, when administered alone, and co-administering to the subject, a subclinical dose of human interferon γ. In one embodiment of the method, for the treatment of viral infection or cancer, the interferon administered is interferon α and the interferon α may include a mixture of interferon α subtypes.

In one embodiment of the method for the treatment of viral infection or cancer, the interferon administered is interferon β.

In another embodiment of the method, interferon γ is co-administered with the interferon α or human interferon β, by administration of a composition containing a mixture of human interferon γ and human interferon α or human interferon β, in a mole ratio of between 1:1 to 1:1000 interferon γ to interferon α or human interferon β. The composition, in one embodiment is formulated in an injectable solution, and is administered by injection; in another embodiment, the composition is formulated for aerosloization, and is administered by inhalation.

In third aspect of the invention provides a composition for use in human therapy, comprising a mixture of human interferon α and human interferon β, in a mole ratio of between 1:10 to 10:1, and a pharmaceutically acceptable carrier. In one exemplary embodiment, the mixture contains a mole ratio of human interferon α to human interferon β between 10:1 and 1:1. In other exemplary embodiment, the mixture contains a mole ratio of human interferon α to human interferon β between 1:1 and 1:10.

In one embodiment, the interferon α includes a mixture of interferon α subtypes, and the mole ratio of human interferon β and interferon α is calculated on the basis of the combined mole ratio of all of the subtypes present. The mixture is produced by culuring interferon-producing cells in culture medium, under conditions of stimulated interferon production, and co-purifying interferon α and human interferon β from the culture medium.

The composition can be formulated in a pharmaceutically injectable solution, or formulated for dispersion in a particle aerosol for administration by inhalation, or formulated in a dry powder form.

The invention further provides a method of treating a viral infection in a human subject, comprising administering to the subject, a pharmaceutically effective dose of a mixture of human interferon α and human interferon β, in a mole ratio of between 1:10 to 10:1. The interferon α includes a mixture of interferon α subtypes, and the mole ratio of human interferon β and interferon α is calculated on the basis of the combined mole ratio of all of the subtypes present. In exemplary embodiments the mixture contains a mole ratio of a mixture of human interferon α to human interferon β between 1:10 and 1:1 and a mole ratio of a mixture of human interferon α to human interferon β between 1:1 and 10:1.

For treating hepatitis C or hepatitis B viruses, the mixture is administered by injection or inhalation.

In another aspect of the invention is a method of treating cancer in a human subject, comprising administering to the subject, a pharmaceutically effective dose of a mixture of human interferon α and human interferon β, in a mole ratio of between 1:10 to 10:1. In exemplary embodiments, the mixture contains human interferon α to human interferon β between 1:10 and 1:1. In other exemplary embodiments, the mixture contains a mole ratio of human interferon α to human interferon β between 1:1 and 10:1.

In one embodiment of the method of the invention, the interferon α includes a mixture of interferon α subtypes, and the mole ratio of human interferon β and interferon α is calculated on the basis of the combined mole ratio of all of the subtypes present.

Another aspect of the invention is a method of treating multiple sclerosis in a human subject, comprising administering to the subject, a pharmaceutically effective dose of a mixture of human interferon α and human interferon β, in a mole ratio of between 1:10 to 10:1.

These and other objects and features of the invention will become more fully apparent from the following detailed description of the invention.

### BRIEF DESCRIPTION OF THE FIGURES

FIG. 1 shows the results of a propidium iodide assay for cell viability in parental wild type (WT) and CrmA-expressing (CrmA-#2) MG-63 cells following superinduction and viral induction with Sendai virus.

FIG. 2 shows interferon-beta production in parental wild type (WT) and CrmA-expressing (CrmA-#2) MG-63 cells following superinduction and Sendai virus treatment.

FIG. 3 shows the effect of 0, 2 mM, 4 mM, and 8 mM 2-aminopurine (2-AP; a PKR inhibitor) on interferon-beta production in CrmA-expressing (CrmA-#2) MG-63 cells following superinduction.

FIGS. 4A and 4B show the percentage of viable 6A, A9 and WT cell lines following cytokine induction by Sendai virus (4A) and poly IC (4B), respectively.

FIGS. 5A and 5B show the IFN-alpha levels produced in Namalwa cell transformants 6A, A9 and WT cell lines following treatment with Sendai virus (5A) and poly IC (5B), respectively.

FIG. 6 shows the coexpression of TNF-beta, IL-6 and IL-8 cytokines in cultures of a Namalwa wild type (WT) and a Namalwa PKR-overexpressing cell line Namalwa PKR++41027 cells, subclone: 2A1.D1.G7.C1.A9.

### DETAILED DESCRIPTION OF THE INVENTION

I. Definitions

Unless otherwise indicated, all technical and scientific terms used herein have the same meaning as they would to one skilled in the art of the present invention. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

The term “vector”, as used herein, refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art. A cloning or expression vector may comprise additional elements, e.g., the expression vector may have two replication systems, thus allowing it to be maintained in two organisms, e.g. in human cells for expression and in a prokaryotic host for cloning and amplification. Cloning and expression vectors also typically contain a selectable marker.

As used herein, the term “selectable marker-encoding nucleotide sequence” refers to a nucleotide coding sequence that is capable of expression in mammalian cells and where expression of the selectable marker confers to cells containing the expressed gene the ability to grow in the presence of a selective agent.

As used herein, the term “promoter” refers to a nucleic acid sequence that functions to direct transcription of a downstream gene. The promoter will generally be appropriate to the host cell in which the target gene is being expressed. The promoter together with other transcriptional and translational regulatory nucleic acid sequences (“control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences. A promoter may be constitutive or inducible and may be a naturally occurring, engineered or hybrid promoter. Hybrid promoters combine elements of more than one promoter, are generally known in the art, and are useful in practicing the present invention.

The term “control sequences” refers to DNA sequences necessary for the expression of an operably linked coding sequence in a particular host organism. The control sequences that are suitable for prokaryotes typically include a promoter, optionally an operator sequence, and a ribosome binding site. Eukaryotic cells are known to utilize promoters, polyadenylation signals, and enhancers.

A “heterologous” nucleic acid construct or sequence includes a portion of the sequence which is not native to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous” nucleic acid construct may contain a control sequence/DNA coding sequence combination that is the same as, or different from a control sequence/DNA coding sequence combination found in the native cell.

As used herein, the term “operably linked” relative to a recombinant DNA construct or vector refers to nucleotide components of the recombinant DNA construct or vector that are directly linked to one another for operative control of a selected coding sequence. Generally, “operably linked” DNA sequences are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “gene” means a segment of DNA involved in producing a polypeptide chain, which may or may not include regions preceding and following the coding region, e.g. 5′ untranslated (5′ UTR) or “leader” sequences and 3′ UTR or “trailer” sequences, as well as intervening sequences (introns) between individual coding segments (exons).

“Cells transfected with a vector” refers to cells which have been exposed to a vector, and have taken up the vector, either as a self-replicating genetic element or by integration into the cell genome, in a manner that allows expression of the protein(s) encoded by the vector. The expression may be under the control of a constitutive promoter in the vector, in which case protein expression occurs in the absence of an inducing agent, or under the control of an inducible promoter, requiring the presence of an inducer in the culture medium in order to achieve expression or high level expression of the vector gene.

As used herein, “recombinant” includes reference to a cell or vector, that has been modified by the introduction of a heterologous nucleic acid sequence or that the cell is derived from a cell so modified. Thus, recombinant cells exhibit modified gene expression, such as expression of genes not found in identical form within the native (non-recombinant) cell or expression of native genes that are otherwise abnormally expressed, underexpressed or not expressed at all, as a result of deliberate human intervention.

As used herein, the terms “transformed”, “stably transformed” or “transgenic” with reference to a mammalian cell means the mammalian cell has a non-native (heterologous) nucleic acid sequence integrated into its genome which is maintained through two or more generations.

As used herein, the term “expression” refers to the process by which a polypeptide is produced based on the nucleic acid sequence of a gene. The process typically includes both transcription and translation, but in some cases may refer to transcription in the absence of translation.

The term “cytokine regulatory factor expression” refers to transcription and translation of a cytokine regulatory factor gene, the products of which include precursor RNA, mRNA, polypeptide, post-translation processed polypeptide, and derivatives thereof, and including cytokine regulatory factors from other species such as murine or simian enzymes.

It follows that the term “PKR expression” refers to transcription and translation of a PKR encoding nucleic acid sequence, the products of which include precursor RNA, mRNA, polypeptide, post-translation processed polypeptide, and derivatives thereof, and including PKRs from other species such as murine or simian enzymes.

As used herein, the terms “biological activity” and “biologically active”, refer to the activity attributed to a particular protein in a cell line in culture. It will be appreciated that the “biological activity” of such a protein may vary somewhat dependent upon culture conditions and is generally reported as a range of activity. Accordingly, a “biologically inactive” form of a protein refers to a form of the protein that has been modified in a manner which interferes with a known activity of the protein.

As used herein, the terms “biological activity of a cytokine regulatory factor” and “biologically active cytokine regulatory factor” refer to any biological activity associated with the a particular cytokine regulatory factor or any fragment, derivative, or analog of that cytokine regulatory factor, e.g., enzymatic activity, etc.

As used herein, the terms “normal level of cytokine regulatory factor activity” and “normal level of cytokine regulatory factor expression” refer to the level of cytokine regulatory factor activity or expression, determined to be present in unmodified, uninduced, unprimed or uninfected cells of a particular type, e.g., the parental cell line of a particular type. It will be appreciated that such “normal” cytokine regulatory factor activity or expression, is reported as a range of cytokine regulatory factor activity or expression that is generally observed for a given type of cells which have not been modified by introduction of a cytokine regulatory factor-encoding nucleic acid sequence or selected for cytokine regulatory factor overexpression.

It follows that the terms “biological activity of PKR” and “biologically active PKR” refer to any biological activity associated with PKR, or a fragment, derivative, or analog of PKR, such as enzymatic activity, specifically including autophosphorylation activity and kinase activity involving phosphorylation of substrates such as eukaryotic translation initiation factor 2 (eIF-2) and transcription factors such as NF-κB.

The range of “normal” cytokine regulatory factor activity or expression may vary somewhat dependent upon culture conditions. For example, the U937 cell line may have a normal range of cytokine regulatory factor activity which differs from the normal range of cytokine regulatory factor activity for the Namalwa cell line. It follows that overexpression of cytokine regulatory factor means an expression level which is above the normal range of cytokine regulatory factor expression generally observed for a given type of cells which have not been modified by introduction of a cytokine regulatory factor-encoding nucleic acid sequence or selected for cytokine regulatory factor overexpression, are unstimulated (not primed or induced) and are uninfected.

Accordingly, “overexpression” of cytokine regulatory factor means a range of cytokine regulatory factor activity, expression or production which is greater than that generally observed for a given type of cells which have not been modified by introduction of a vector comprising the coding sequence for PKR or selected for PKR overexpression, are unstimulated (not primed or induced) and are uninfected.

In one preferred aspect, cytokine regulatory factor overexpression means a level of cytokine regulatory factor activity, expression or production that is at least 125% (1.25-fold or 1.25×), preferably at least 150%, 200%, 300% or 400%, or 500% or more greater than the normal level of cytokine regulatory factor activity, expression or production for the same cell line under the particular culture conditions employed. In other words, a cell line that over expresses a cytokine regulatory factor typically exhibits a level of cytokine regulatory factor production or expression that is at least 1.25-fold and preferably 1.5-fold (1.5×), 2-fold (2×), 3-fold (3×), 4-fold (4×), 5-fold (5×) or more greater than the level of cytokine regulatory factor expression or production typically exhibited by the same type of cells which have not been selected, modified, primed or treated in a manner effective result in cytokine regulatory factor overexpression.

In some cases, a cell line that over expresses a cytokine regulatory factor such as PKR exhibits a level of cytokine regulatory factor expression or production that is 10-fold (10×) or more greater than the level of cytokine regulatory factor expression or production typically exhibited by the same type of cells under the particular culture conditions employed and which have not been selected, modified, primed or treated in a manner effective result in cytokine regulatory factor overexpression.

As used herein, the terms “normal level of cytokine” and “normal level of protein”, relative to activity, expression, and production, refer to the level of cytokine or other protein activity, expression or production, determined to be present in parental cells of a particular type which have not been selected, modified, primed or treated in a manner effective result in cytokine regulatory factor overexpression. Examples include, a wild type (“parental”) cell line which has not been selected, primed or treated in a manner to result in enhanced cytokine regulatory factor activity, expression or production, and a cell line which does not comprise an introduced cytokine regulatory factor coding sequence. It will be appreciated that such “normal” cytokine or other protein activity, expression, or production, is reported as a range of activity, expression, or production, typically observed for a given type of cells and may vary somewhat dependent upon culture conditions.

The terms an “enhanced level of” and “above normal level of” relative to cytokine or protein activity, expression or production may be used interchangeably. The terms refer to a level of cytokine or protein activity, expression or production that is at least 125%, (1.25-fold or 1.25×), preferably at least 150%, 200%, 300% or 400%, or 500% or more greater than the level of cytokine or protein activity, expression or production exhibited by parental cells of the same cell line under the particular culture conditions employed, where the parental cells have not been selected, modified, primed or treated in a manner effective to result in an increase in cytokine or protein activity, expression, or production. In some cases, the increase in cytokine or protein activity, expression, or production is 10-fold (10×) or more greater than that of the parental cell line.

As used herein, the term “inhibit apoptotic cell death”, means to partially or completely inhibit the cell death process over the time period a cell line is cultured for the purpose of cytokine or other protein expression or production. Such inhibition generally means the amount of apoptotic cell death is decreased by at least 20%, preferably by at least 50% and more preferably by 80% or more relative to the amount of apoptotic cell death observed in a cell line which has not been modified in a manner effective to inhibit apoptosis.

The term “proteins that inhibit apoptosis” refers to proteins that, when expressed in a cell, inhibit apoptosis, and in particular, apoptosis associated with cytokine regulatory factor overexpression and/or cytokine induction. Examples of proteins effective to inhibit apoptosis include, but are not limited to, CrmA, Bcl-2a, Bcl-X_{L}, a modified from of eukaryotic translation initiation factor 2 alpha (eIF-2 alpha) and eukaryotic translation initiation factor (eIF-3), a modified form of Fas-associated death domain (FADD), a modified form of Bcl-X_{S}, a modified form of Bcl-2-homologous antagonist/killer (BAK) and a modified from of BAX, preferably Bcl-2a or Bcl-X_{L.}

“Overexpression” of CrmA, Bcl-2 or Bcl-X_{L}, respectively, means a range of CrmA, Bcl-2 or Bcl-X_{L} activity or expression which is greater than that generally observed for a given type of cells which have not been transfected with a vector encoding CrmA, Bcl-2 or Bcl-X_{L}, and which are stimulated to undergo apoptosis.

“Cytokines” refers to a group of low-molecular-weight regulatory proteins that regulate the intensity and duration of the immune response by exerting a variety of effects on lymphocytes and other immune cells.

“Cytokine-producing cells” refers to cells, typically blood cells, that secrete cytokines in vivo, and also in cell culture. Such cells include monocytes, macrophages, dendritic cells, B cells, endothelial cells, epithelial cells, T_{H}1 and T_{H}2 (T-helper cells), NK (natural killer) cells, eosinophils mast cells, bone-marrow cells, fibroblasts, keratinocytes, osteoblast-derived cells, melanocytes, platelets, various other immune system cells, pancreatic parenchymal cells, glial cells and tumor cells derived from such cell types.

The terms “modifying” and the term “cell line modification” as used herein relative to a cultured human cell line refer to introducing a heterologous nucleic acid sequence that encodes a cytokine regulatory factor and/or a heterologous nucleic acid sequence that encodes an anti-apoptotic protein into a parental human cell line. The coding sequence in the heterologous nucleic acid construct may be of heterologous or autologous origin.

“Selecting” cytokine regulatory factor overexpressing cells generally refers to subcloning, screening and selecting for cells that overexpress one or more cytokine regulatory factors and growing the cells to produce a cytokine regulatory factor overexpressing cell line.

Screening typically includes functional assays for biological activity, protein assays and assays for cytokine regulatory factor mRNA, as further described below.

The term “priming” as used herein relative to a cytokine regulatory factor overexpressing cell line typically refers to exposing the cells to any of a number of agents, such as phorbol myristate acetate (PMA) or interferon-β.

The terms “induction” and “inducing” as used herein relative to a cytokine regulatory factor overexpressing cell line typically refer to exposing cells to a microbial inducing agent, such as Sendai virus, encephalomyocarditis virus, Herpes simplex virus or Newcastle Disease Virus; or exposing the cells to at least one non-microbial inducing agent selected from the group consisting of poly(I):poly(C) (poly IC), or poly r(I):poly r(C) (poly rIC), heparin, dextran sulfate, cyclohexamide, Actinomycin D, sodium butyrate, calcium ionophores, phytohemagglutinin (PHA), lipopolysaccharide (LPS) and derivatives thereof, such as 3-deacyl LPS, and chondroitin sulfate. Specific examples of inducing agents include, surface roughness for the induction of prostaglandin E2, titanium particles for induction of TGF-beta, silicon nitride for induction of TNF-alpha, hypoxia for induction of EPO and VEGF, and IL-1-alpha, TNF-alpha and TNF-beta for induction of osteoprotegerin.

The terms “treating” and “treated”, as used herein relative to a cytokine regulatory factor overexpressing cell line generally refers to induction, but may be used with reference to priming and/or induction and/or exposure to an additional agent, e.g., DEAE Dextran.

The term “cytokine mixture” as used herein refers to a composition comprising two or more cytokines produced by a human cell line.

The terms “treating”, “treatment” and “therapy” as used herein relative to a human subject or patient refer to curative therapy, prophylactic therapy, and preventative therapy.

II. Cytokine Regulatory Factors

A number of factors are known to be involved in the induction and/or enhanced expression of cytokines in cells, e.g., human cells. These factors include cytokine- and other protein-specific transcriptional regulatory factors, e.g. interferon regulatory factors (IRF-1, IRF-3 and IRF-7), cytokine receptors, nuclear factor κB (NF-κB), activator protein-1 (AP-1), nuclear factor IL-6 (NF-IL6), and in particular, PKR.

Enhancing the expression or activity of any of these factors will generally result in higher than normal expression of one or more cytokine-encoding genes. PKR is used as herein as an example of a protein capable of regulating cytokine and other protein expression; however, it will be understood that the invention contemplates any of a number of cytokine and protein enhancing factors (designated herein as “cytokine regulatory factors” or “CRF”), e.g., protein kinase C (PKC) inducers, TNF-α, GM-CSF, EGF and PDGF, G-CSF, TGF, TNF-alpha or TNF-beta, IL-1, IFNs (IFN-alpha, IFN-beta, IFN-gamma) or chemokines (IL-8, Macrophage inflammatory proteins [MIP-1a & -1b] and monocyte chemotactic proteins [MCPs]); other cellular signaling factors such as PMA, calcium ionophores, sodium butyrate or endotoxin; polyl: C, double-stranded RNA or viral analogs; PHA, cellular stress signals that can activate PKR, including heat shock, pathogen infection, e.g. viral infection; and any factor that enhances expression of a cytokine regulatory factor resulting in enhanced cytokine production.

By increasing the expression/activity of a cytokine regulatory factor in human cells, cytokine production can be increased. Human cell cultures that express a higher constitutive level of the cytokine regulatory factor, or in which cytokine regulatory factor expression can be induced to higher levels are therefore useful for the production of mixtures of cytokines.

The methods of the invention rely on the use of cells that overexpress a cytokine regulatory factor, with no particular method of cytokine regulatory factor overexpression required.

Various functions have been attributed to PKR, including, phosphorylation of eukaryotic initiation factor-2 (eIF-2alpha), which, once phosphorylated, leads to inhibition of protein synthesis (Hershey, et al., 1991). This particular function of PKR has been suggested to be one of the mechanisms responsible for mediating the antiviral and anti-proliferative activities of IFN-alpha and IFN-beta. An additional biological function for PKR is its putative role as a signal transducer, for example, by phosphorylation of IkB, resulting in the release and activation of nuclear factor kB (NF-kB) (Kumar A et al., 1994).

It has previously been demonstrated that PKR mediates the transcriptional activation of IFN expression (Der D and Lau A S, 1995). Consistent with this observation, suppression of endogenous PKR activity by transfecting U937 cells with antisense to PKR or expression of a PKR-deficient mutant resulted in diminished induction of IFN in response to viral infection (Der D and Lau A S, 1995).

It has also been demonstrated that cells transfected with a PKR-encoding nucleic acid sequence exhibit enhanced interferon production, as described in co-owned U.S. Pat. No. 6,159,712.

It has also been suggested that PKR may function as a tumor suppressor and inducer of apoptosis. (See, e.g., Clemens M J et al., 1999; Yeung, Lau et al, 1996; Koromilas et al., 1992). Recent results indicate that expression of an active form of PKR triggers apoptosis, possibly through upregulation of the Fas receptor (Donze O, et al., 1999).

The invention employs cytokine-producing cells that overproduce a cytokine regulatory factor such as PKR by virtue of introduction of a cytokine regulatory factor-encoding nucleic acid sequence or by culturing non-transformed cells, or cells transformed with an apoptosis-inhibiting gene only, under conditions which produce above-normal levels of one or more endogenous cytokine regulatory factors. This may be accomplished by selection, priming and/or further treatment, such as induction.

With respect to PKR, additional approaches to enhanced production/expression include inactivation or decreasing the levels of the PKR-inhibiting factor, p58 which normally inhibits PKR activity. Mutation, modification or gene-targeting ablation of p58 has been shown to result in enhanced PKR activity (Barber, G. N. et al., 1994). Further, natural, synthetic or recombinant activators of PKR that can enhance the expression of PKR, e.g., the PKR activator protein, PACT (Patel, R. C. and Sen, G. C., 1998), may be employed.

III. Methods and Compositions of the Invention

A. Combinations of Cytokines

Cytokines elicit their biological activities by binding to their cognate receptors followed by signal transduction leading to stimulation of various biochemical processes. In some cases, the expression of such receptors is regulated by specific signals. A cytokine may be involved in positive or negative feedback loops and thereby regulate the expression of the receptor for the same or a different cytokine. Such receptors may be the same type of cell that produces the cytokine or a different type of cell. Cytokines serve to mediate and regulate immune and inflammatory responses.

It will be appreciated that the cellular source of cytokines is a distinguishing characteristic of each individual cytokine and that an individual cytokine may be produced by multiple diverse types of cells. In addition, a given cytokine (1) may act on more than one type of cells, (2) may have more than one effect on the same cell, (3) may have an activity shared with another cytokine, and (4) may influence the synthesis or effect of other cytokines, e.g., by antagonizing, or synergizing the effects thereof.

The methods described herein find utility in the production of a mixture of two or more cytokines for therapeutic uses, and in particular, a cytokine mixture for use in treating cancer, viral infection, or inflammation, as detailed in co-owned U.S. application Ser. No. 09/660,468, expressly incorporated by reference herein.

Exemplary cytokines the expression of which may be increased using the methods of the invention include, but are not limited to, interferons (α, β and γ), interleukins (IL-1α, IL-1β, IL-1ra, IL-2 and IL-4 through 13), tumor necrosis factors alpha and beta (TNF-β) and their respective soluble receptors (sTNF-R), the colony stimulating factors (granulocyte colony stimulating factor, G-CSF; granulocyte-macrophage colony stimulating factor, GM-CSF; and IL-3), the angiogenic factors (fibroblast growth factor, FGF; vascular endothelial growth factor, VEGF; and platelet-derived growth factors 1 and 2 (PDGF-1 and -2) and the anti-angiogenic factors (angiostatin and endostatin).

In summary, the invention provides a cytokine mixture produced by a cell line characterized by expression of a cytokine regulatory factor and/or expression of an anti-apoptotic protein which is cultured under appropriate conditions, modified, selected, primed and/or treated in a manner effective to result in production of two or more cytokines. The invention further provides mixtures of cytokines produced by such a cell line. In a preferred approach, the cytokines are produced by the cell line, secreted into the medium and isolated (purified from unwanted components also present in the cell culture medium).

Preferred mixtures include, but are not limited to, (1) interferon alpha (IFN-α) and interferon beta (IFN-β); (2) interferon alpha, interferon beta and interferon gamma (IFN-γ); (3) interferon alpha and interferon gamma; (4) interferon beta and interferon gamma; (5) one of interferon alpha, interferon beta and interferon gamma plus tumor necrosis factor-alpha (TNF-α); (6) one of interferon alpha, interferon beta and interferon gamma plus TNF-beta (TNF-β); (5) one of interferon alpha, interferon beta and interferon gamma plus interleukin-2 (IL-2); (6) interferon gamma and interleukin-1b (IL-1b); (7) interferon gamma and macrophage colony stimulating factor (M-CSF); (8) interferon gamma, interleukin-4 and tumor necrosis factor-alpha; (9) interleukin-2 and interleukin-12; (10) one of interleukin-2, interleukin-4 or interleukin-6 and granulocyte-macrophage colony stimulating factor (GM-CSF); (11) granulocyte-macrophage colony stimulating factor, interleukin-2 and interferon alpha or interferon beta.

One preferred anti-cancer or anti-tumor composition includes two or more cytokines selected from among IL-1-alpha, IL-1-beta, IL-2, IL4, IL-6, IL-12, IL-15, IFN-alpha, IFN-beta, IFN-gamma, oncostatin, TNF-alpha, TNF-beta, GM-CSF, G-CSF, NKEF, NKSF, TRAIL and M-CSF, more preferably selected from among IL-2, IL-12, IL-15, IFN-alpha, IFN-beta, TNF-alpha, natural killer cell enhancement factor (NKEF), natural killer cell stimulatory factor (NKSF), TNF-related-apoptosis-inducing-ligand (TRAIL) and GM-CSF. The composition is preferably treated to remove cytokine(s) selected from among IL-3, IL-5, IL-7, IL-8, IL-9, IL-10, IL-11, IL-1 and TGF-beta.

For use in treating viral infection, the composition preferably includes two or more cytokines selected from among IFN-alpha, IFN-beta, IFN-gamma, TGF-beta, IL-3, IL-7, IL-8, IL-12, and GM-CSF, more preferably selected from among IFN-alpha, IFN-beta, TGF-beta, IL-8, IL-12, and GM-CSF. The composition is preferably treated to remove cytokine(s) selected from among IL-1, IL-2, IL-4, IL-5, IL-6, IL-9, IL-10, IL-11, IL-13, TNF-alpha, TNF-beta, and oncostatin.

For use in treating an inflammatory condition, the composition preferably includes two or more cytokines selected from among IL-4, IL-5, IL-6, IL-10, IL-11, IL-13, IL-1i, IFN-beta, TGF-beta and IFN-gamma, and preferably selected from among IL-4, IL-5, IL-6, IL-10, IFN-beta, IFN-gamma and TGF-beta. The composition is preferably treated to remove from the composition, cytokine(s) selected from among IL-1, IL-2, IL-3, IL-7, IL-8, IL-9, IL-12, TNF-alpha, TNF-beta, TGF-beta, and oncostatin.

The cytokine mixtures or compositions of the invention find utility in the treatment of cancer, viral infection or inflammation. In general, the total cytokine dose administered is adjusted so that any one cytokine component is administered at a lower dose, e.g., 10%-50% of the normal dose of that cytokine when administered alone.

B. Cells and Culture Conditions for Production of Cytokine Mixtures

The invention relies on human-cytokine producing cells that are selected for their ability to produce a desired mixture of cytokines, e.g., a mixture suitable for cancer treatment, treatment of viral infection, or treatment of inflammation.

Thus, the present invention provides cell lines comprising cells that have been selected, modified, primed and/or primed and induced in a manner effective to result in enhanced production of mixtures of cytokines relative to the corresponding parental cell line (which is unmodified, unselected, unprimed and uninduced).

Examples of parental cell lines for use in the production of mixtures of cytokines include, but are not limited to B cells (for example Namalwa, 293, CCRF-SB or Raji cells), monocytic cells (U937, THP-1) Flow 1000 cells, Flow 4000 cells, fibroblasts (MRC-5, WI-38, FS-4, FS-7, T98G and MG-63 cells), T cells (CCRF-CEM, and Jurkat cells) and other cells.

Examples of parental cell lines for use in the production of mixtures of cytokines include, but are not limited to, cells of the monocyte/macrophage lineage, lymphocytic lineage cells including T- and B-cells, mast cells, fibroblasts, bone marrow cells, keratinocytes, osteoblast-derived cells, melanocytes, endothelial cells, platelets, various other immune system cells, lung epithelial cells, pancreatic parenchymal cells, glial cells and tumor cells derived from such cell types. Major cellular sources for a variety of cytokines are provided in Table 1, below. Cell lines derived from these cells are suitable candidates for use in producing cytokine mixtures using the methods described herein.

TABLE 1
Cellular Source of Various Cytokines

| | Molecular | |
|---|---|---|
| Cytokine | Weight (kD) | Major cellular sources |
| Interferons | | |
| IFN-α (>12 subtypes) | 16-20 | macrophages, fibroblasts, |
| | | lymphoblastoid cells |
| IFN-β | 20 | fibroblasts macrophages, |
| | | epithelial cells |
| IFN-γ | 20-25 | T-cells, NK cells, |
| | | dendritic cells |
| Tumor Necrosis Factors | | |
| TNF-α | 17 | monocyte/macrophages, |
| | | fibroblasts, T-cells |
| TNF-β (lymphotoxin) | 25 | T-cells, B-cells |
| Interleukins | | |
| IL-1α & IL-1β | 17.5 | monocyte/macrophages, |
| | | endothelial cells, fibroblas |
| | | keratinocytes |
| IL-2 | 15-17 | T-cells |
| IL-4 | 15-19 | T-cells, mast cells |
| IL-5 | 50-60 | T-cells, mast cells |
| IL-6 | 26 | monocyte/macrophages, |
| | | fibroblasts, T-cells |
| IL-7 | 25 | bone marrow cells |
| IL-8 | 6-8 | monocytes, fibroblasts, |
| | | chondrocytes, endothelial c |
| | | keratinocytes |
| IL-9 | 32-39 | T-cells |
| IL-10 | 19 | T-cells |
| IL-11 | 23 | bone marrow cells |
| IL-12 p35 & p40 | 35, 40 | B-cells, lymphoblastoid cells |
| Colony Stimulating Factors | | |
| IL-3 | 20-26 | T-cells, mast cells |
| G-CSF | 20 | monocyte/macrophages, |
| | | fibroblasts, endothelial cel |
| GM-CSF | 22 | T-cells, fibroblasts, |
| | | endothelial cells |
| M-CSF | 70-90 | monocytes, fibroblasts, |
| | | endothelial cells |
| Growth factors | | |
| Epidermal growth factor | 6 | macrophages |
| Fibroblast growth factors | 14-18 | platelets, macrophages, |
| (acidic & basic) | | endothelial cells |
| Platelet-derived growth | 14-18 | platelets, monocyte/ |
| factors 1 & 2 | | macrophages, endothelial cells |
| TGF-α | 5-8 | macrophage |
| TGF-β | 25 | platelets, monocyte/ |
| | | macrophages, T-cells, |
| | | fibroblas endothelial cells |
| Chemokines | | |
| Macrophage inflammatory | 8 | monocytes, fibroblasts, T cells |
| proteins 1α, 1β | | |
| RANTES | 9.5 | T-cells |

Human cells suitable for producing a mixture of cytokines for use in treating cancer (including solid tumors, melanomas, leukemias, and other types of cancers or neoplasms) include B-lymphocytes (B-cells), monocytic cells, and T helper cells. Examples of isolated B-cell parent cell lines that are suitable for in vitro culturing are Namalwa, 293 and Raji cells. Suitable monocytic parent cell lines are U937 and THP-1 cells. T cells, including T-helper cells 1 and 2 (T_{H}1, T_{H}2) that are available for in vitro culturing include Jurkat, and CEM cells.

Human cytokine-producing cells suitable for producing a mixture of cytokines suitable for use in treating viral infection (including infection by HIV, hepatitis viruses, such as HBV, and HCV virus, and other human-pathogenic viruses) include, generally B-lymphocytes (B-cells) and fibroblast cell lines. Examples of isolated B-cell parent cell lines that are suitable for in vitro culturing are given above. Suitable fibroblast parent cell lines are MRC5, HFF, and WI-38 cells.

Human cytokine-producing cells suitable for producing a mixture of cytokines suitable for use in treating inflammation (including asthma, allergies, and rheumatoid arthritis) generally T-cells, including T-helper cells, as exemplified above.

In general, U937 cells are preferred for production of FGF and sTNF-R; Jurkat cells are preferred for production of IL-3 and TNF-beta; fibroblasts are preferred for production of FGF and angiostatin; U937 cells are preferred for production of TNF-alpha, IFN-alpha, IL-6 and homologues thereof; CD4 expressing cells including Jurkat and HUT are preferred for production of TNF-beta; and T and B-cells including Jurkat and Namalwa are preferred for production of IL-8 and homologues thereof.

Thus, the present invention provides cell lines comprising cells which have been selected, modified, primed and/or primed and induced in a manner effective to result in enhanced production of mixtures of cytokines as compared to the corresponding parental cell line.

The selected cells are cultured under conditions of cytokine regulatory factor and cytokine overexpression. Cells useful for the production of mixtures of cytokines are cultured under conditions typically employed to culture the parental cell line. Generally, the cells are cultured in a standard medium containing physiological salts and nutrients, such as standard cell culture media RPMI, MEM, IMEM DMEM, or F12 which may be supplemented with 0.1-10% serum, such as fetal bovine serum. Alternatively, serum free and/or animal-derived protein-free or protein-free medium may be used, a number of examples of which are commercially available such as Pro293 for example. Culture conditions are also standard, e.g., cultures are incubated at 37° C. in stationary or roller cultures until desired levels of cytokine production is achieved. For large scale production, fermentors may be used with cells grown in batch or a perfusion mode. An exemplary medium and culture conditions for cytokine production from Namalwa cells is described in Example 3.

In general, preferred culture conditions for a given cell line may be found in the scientific literature and/or from the source of the cell line such as the American Type Culture Collection. Preferred culture conditions for primary cell lines, such as fibroblasts, B-cells, T-cells, endothelial cells, dendritic cells, and monocytes are also generally available in the scientific literature. After cell growth has been established, the cells are exposed to conditions effective to cause or permit the overexpression of one or more cytokine regulatory factors, primed and/or primed and induced for enhanced production of mixtures of cytokines.

In cases where an exogenously provided cytokine regulatory factor-encoding nucleic acid sequence is under the control of an inducible promoter, the appropriate inducing agent, e.g., a metal salt or antibiotic, is added to the medium at a concentration effective to induce cytokine expression/production.

IV. Enhanced Cytokine Production

A. Enhanced Cytokine Regulatory Factor Expression

By increasing the expression/activity of a cytokine regulatory factor, such as PKR in human cells, the production of mixtures of human cytokines can be increased. Human cell cultures that express a higher constitutive level of a cytokine regulatory factor, or in which cytokine regulatory factor expression can be induced to higher levels are therefore useful for the production of mixtures of cytokines.

Once a cell line that over expresses or overproduces a cytokine regulatory factor is obtained that cell line may be further primed and/or induced in a manner effective to result in an increase in cytokine production.

In one preferred approach, the method comprises (a) culturing human cells capable of overexpression of a cytokine regulatory factor; (b) introducing a heterologous nucleic acid construct comprising the coding sequence for a cytokine regulatory factor, or an analog or homologue thereof into the cells, under conditions sufficient to overexpress the cytokine regulatory factor; selecting or screening for cells that have incorporated the heterologous nucleic acid and (c) priming; and (d) treating the cells as appropriate to induce the expression of a cytokine gene.

In one preferred embodiment, the cytokine-regulatory factor is PKR and the cells are inducible for PKR overexpression. In a preferred aspect, cells capable of over expressing PKR are primed and/or induced in a manner which results in a higher level of PKR expression or production.

The combination of cell line selection, modification, culture conditions, priming or priming and induction provided by the present invention results in significantly enhanced cytokine production by a given cell line, e.g., an increase that represents an increase of at least 200% (2-fold or 2×), 250% (2.5-fold or 2.5×), 400% 3-fold or 3×), 400% (4-fold or 4×), 500% (5-fold or 5×), and preferably 1000% (10-fold or 2×), or more cytokine production or expression relative to the level of cytokine production or expression exhibited by the same cell line under the same culture conditions absent selection, modification, priming and induction, as described herein. In some cases, the methods of the invention result in an increase in cytokine production that is 100-fold (100×) to 1000-fold (1000×) or more.

Accordingly, one aspect of the present invention pertains to an isolated population of cells, i.e. a cell line, which expresses or produces a mixture of cytokines. The term “population” as used herein refers to a group of two or more cells, which have been derived from a single parental cell.

Problems typically associated with production of cytokines in cell culture, for example low yield from non-recombinant mammalian systems, improper glycosylation, lack of appropriate post translational modification or misfolding of proteins produced in microbial systems are eliminated in the methods of the present invention.

B. Inhibition of Apoptosis

Apoptosis or programmed cell death is a cell-intrinsic suicide process whereby unwanted individual cells undergo a genetically determined program, culminating in chromosomal DNA fragmentation, degradation of RNA and eventual cell death (reviewed in Orrenius 1995; Stellar 1995; Vaux 1993). Once committed to apoptosis, the cells undergo new rounds of protein synthesis and various morphological/physiological changes including cytoplasmic condensation, nuclear chromatin condensation, membrane blebbing, and eventual DNA degradation, detected as a characteristic oligonucleosomal ladder.

TNFs, as prototypes proinflammatory cytokines, are cytotoxic proteins produced by activated immune cells during the processes of pathogen elimination, antiviral activities, and tumor destruction. However, high levels of TNF-alpha in vivo can be detrimental since TNF-alpha induces metabolic disturbances, wasting, and suppression of hematopoiesis. At the cellular level, TNF-alpha induces production of superoxide radicals, activation of lysosomal enzymes (Larrick, et al., 1990; Liddil, et al., 1989), and fragmentation of DNA by the activation of endonuclease activity (Rubin, et al, 1988), leading to apoptosis.

PKR is known to play a role in the TNF-α signaling pathway and in the induction of apoptosis. It has been shown that U937 cells which over express PKR, also exhibit increased apoptosis. (See, e.g., Yeung M C, et al., 1996 and Yeung M C, et al., 1999.)

Individual proto-oncogenes that have been associated with apoptosis may be expressed in cells undergoing apoptosis, and modulation of expression of individual proto-oncogenes has been observed to affect the process. Exemplary proto-oncogenes include c-myc, Fas (APO-1), p53, and Bcl-2 in addition to other genes such as ced-3, ced4, ced-9 and Ice (Stellar, 1995; Cohen, 1993).

By inhibiting apoptosis, the cell lines described herein have a longer lifespan in culture and exhibit an increase in biosynthesis of cytokines and/or an increase in the time over which the cells function to produce cytokines.

Accordingly, in one preferred aspect of the invention, a selected gene protein capable of inhibiting apoptosis, e.g., CrmA, Bcl-2a, Bcl-X_{L} or a homologue thereof is overexpressed in the host cell resulting in a suppression or delay in apoptotic cell death.

In other cases, a “modified form of”, a protein associated with apoptosis, is expressed in a cell. Suppressing the expression of a gene encoding a protein associated with apoptosis may result in suppression or delay of apoptotic cell death and can be effected by methods including, but not limited to, mutation of an endogenous gene, homologous recombination or site directed mutagenesis, gene deletion or gene ablation or any method effective to result in the abolition or altered expression of a gene which encodes a protein associated with apoptosis.

Proteins associated with apoptosis include, but are not limited to eIF-2a or eIF-2alpha, eIF-3, FADD, Bcl-X_{S}, BAK, BAX, and the like. A “modified form of” a protein means a derivative or variant form of the native protein that generally has a derivative polypeptide sequence containing at least one amino acid substitution, deletion or insertion, with amino acid substitutions being particularly preferred. The amino acid substitution, insertion or deletion may occur at any residue within the polypeptide sequence, which interferes with the biological activity of the protein. The corresponding nucleic acid sequence which encodes the variant or derivative protein is considered to be a “mutated” or “modified form of” the gene or coding sequence therefore, and is included within the scope of the invention.

By way of example, suppression of the apoptotic cell death process in human cell culture may be achieved by: (1) overexpression of a protein capable of inhibiting apoptosis, examples of which include, but are not limited to CrmA, Bcl-2a and Bcl-X_{L} or a homologue thereof; (2) suppression of eIF2-alpha (GenBank Accession No. A 457497) phosphorylation, e.g., by overexpression of a mutant form of eIF2-alpha or eukaryotic translation initiation factor (eIF-3), prepared by mutation of the respective endogenous gene using homologous recombination or site directed mutagenesis (thereby inhibiting the downstream substrates of PKR); (3) suppression of endogenous FADD activity, e.g., by overexpression of a mutant form of FADD, prepared by mutation of the endogenous FADD gene using homologous recombination or site directed mutagenesis; or (4) use of a transdominant mutant, by mutation of an endogenous gene for one or more pro-apoptotic counterparts of Bcl-2a, e.g. BAX (GenBank Accession No. L22473), BAK (GenBank Accession No. BE221666), and/or Bcl-X_{S} (GenBank Accession No. L20122) by homologous recombination or site-directed mutagenesis, or by gene ablation or gene deletion of one or more of BAX, BAK, and Bcl-X_{S}.

Cell death may be detected by staining cells with propidium iodide (PI), or by use of assays specific to apoptotic cell death, e.g., by staining with annexin V (Vermes, et al., 1995). Necrotic cell death may be distinguished from apoptotic cell death by evaluating the results of a combination of the assays for cell viability, together with microscopic observation of the morphology of the relevant cells.

In one approach, a cytokine regulatory factor overexpressing cell line is provided and transfected with a vector containing an anti-apoptotic gene, such that transfection and selection of cells for anti-apoptotic function is carried out using cells that have already been “stabilized” with respect to cytokine regulatory factor overexpression. In this case, the cytokine regulatory factor overexpressing cell line may be prepared by subcloning and selection or by introduction and expression of a cytokine regulatory factor-encoding nucleic acid sequence.

In an alternative approach, the cells are first transfected with a vector containing an anti-apoptotic gene, then successful transformants may be further transfected with a vector containing a cytokine regulatory factor-encoding nucleic acid sequence. This allows for the second transfection and selection to be carried out using cells that have already been “stabilized” with an anti-apoptotic function. Alternatively, cells that have already been “stabilized” with an anti-apoptotic function may be subcloned and selected for cytokine regulatory factor overexpression.

Methods for enhancing the production of cytokines in cell culture by inhibiting apoptosis associated with cytokine synthesis, particularly under conditions of cytokine regulatory factor overexpression are further described in co-owned U.S. application Ser. No. 09/772,109, expressly incorporated by reference herein.

V. Increasing Endogenous Cytokine Regulatory Factor Activity, Expression and/or Production

In accordance with the present invention, it has been discovered that cell lines capable of expressing one or more cytokine regulatory factors and mixtures of cytokines, may be subjected to limiting dilution cloning (referred to herein as “subcloning”), screened for enhanced cytokine regulatory factor activity and/or mRNA and/or protein expression, further subcloned and selected for enhanced cytokine activity and/or expression, as further detailed below. Exemplary cytokines which may be used as markers for such enhanced cytokine regulatory factor overexpression, include but are not limited to TNF-alpha and beta, IL-2, IL-6, IL-8, IL-10, GM-CSF, and IFNs.

In practicing the method, a cell line capable of expressing one or more cytokine regulatory factors and one or more cytokines (designated herein as the “parental cell line”) is identified and subjected to limiting dilution cloning of single cells, using standard methods routinely employed by those of skill in the art. In general, the subcloning step is carried out at least 1 times, and typically 3 to 5 times in succession in 96 well plates. Subclones are grown to obtain a population of approximately 0.3 to 0.5 million cells/ml using culture conditions typically employed to culture the parental cell line. The subclones are then assayed for cytokine regulatory factor and cytokine expression.

Exemplary assays for cytokine regulatory factor expression and/or production include functional assays for biological activity, protein assays such as Western blot and assays for cytokine regulatory factor mRNA such as RT-PCR (reverse transcriptase polymerase chain reaction) and Northern blotting, dot blotting, or in situ hybridization using an appropriately labeled probe based on the cytokine regulatory factor-encoding nucleic acid sequence.

Subclones that exhibit a level of cytokine regulatory factor expression or production that is at least 2-fold (2×), and preferably 3-fold (3×), 4-fold (4×), 5-fold (5×) or more greater than the level of cytokine regulatory factor expression or production of the parental cell line are selected. In some cases, such selected subclones exhibit a level of cytokine regulatory factor expression or production that is 10-fold (10×) or more the level of cytokine regulatory factor expression or production of the parental cell line.

Typically, selected subclones are primed by exposure to a priming agent prior to being treated in a manner effective to result in enhanced cytokine regulatory factor and cytokine production. Exemplary priming agents are further described below. Selected subclones are then induced in a manner effective to result in enhanced cytokine regulatory factor expression and enhanced production of mixtures of cytokines.

VI. Increasing Cytokine Regulatory Factor by Expression of a Heterologous Nucleic Acid Construct in a Cell

The invention also provides host cells that have been transduced, transformed or transfected with an expression vector comprising a cytokine regulatory factor-encoding nucleic acid sequence. The culture conditions, such as temperature, pH and the like, are those previously used for the parental host cell prior to transduction, transformation or transfection and will be apparent to those skilled in the art.

In one approach, a human cell line is transfected with an expression vector having a promoter or biologically active promoter fragment or one or more (e.g., a series) of enhancers which functions in the host cell line, operably linked to a DNA segment encoding one or more cytokine regulatory factors, such that the one or more cytokine regulatory factors are overexpressed in the cell line.

In one approach, parental cells are transfected with an expression vector comprising a cytokine regulatory factor-encoding nucleic acid sequence under the control of a constitutive or inducible promoter, such that culturing the cells in a suitable growth medium leads to overexpression of the cytokine regulatory factor nucleic acid sequence. The cells may also be transfected with a second vector that expresses a protein that inhibits apoptosis in the cells under conditions of cytokine regulatory factor overexpression and/or cytokine production.

The examples describe exemplary vectors and transfection methods for obtaining human cytokine-producing cells suitable for use in the invention. The cells are sequentially transfected with the vector containing a cytokine regulatory factor and an anti-apoptotic gene, with successful transformants selected prior to the second transfection. This allows for the second transfection and selection to be carried out with cells that have already been “stabilized” for expression of a cytokine regulatory factor or anti-apoptotic gene. The vector construction and transfection conditions are conventional, and known to those skilled in the art. In particular, it is well known, in such vector constructions, to obtain suitable plasmids or other vectors, e.g., from commercial sources, capable of being introduced into and replicating within selected human cells, where the plasmids may also be equipped with selectable markers, insertion sites, and suitable control elements, such as termination sequences. Exemplary coding sequences for a cytokine regulatory factor, the PKR gene, and for an anti-apoptotic gene, Bcl-X_{L}, are referenced in Example 1, and can be obtained from the GenBank as cited.

Appropriate cloning and expression vectors for use in human cells are described in Sambrook et al. (1989) Molecular Cloning: A Laboratory Manual (Second Edition), Cold Spring Harbor Press, Plainview, N.Y. and Ausubel F M et al. (1989) Current Protocols in Molecular Biology, John Wiley & Sons, New York, N.Y., expressly incorporated by reference herein. Exemplary promoters include both constitutive promoters and inducible promoters, examples of which include a CMV promoter, an SV40 early promoter, an RSV promoter, an EF-1alpha promoter, a promoter containing the responsive element (TRE) and the metallothienein promoter.

A. Vectors

Natural or synthetic polynucleotide fragments encoding one or more cytokine regulatory factors (“cytokine regulatory factor-encoding nucleic acid sequences”) or anti-apoptotic genes may be incorporated into heterologous nucleic acid constructs or vectors, capable of introduction into, and replication in, a human cell. The vectors and methods disclosed herein are suitable for use in host cells for the expression of one or more cytokine regulatory factors or anti-apoptotic genes. Any vector may be used as long as it is replicable and viable in the human cells into which it is introduced. Large numbers of suitable vectors and promoters are known to those of skill in the art, and are commercially available.

Vectors are the means by which DNA is delivered to the target cell. Methods known in the art for delivery of nucleic acid constructs into mammalian cells include viral methods using adenoviral vectors, retroviral vectors, or adeno-associated viral vectors. In general, the efficiency of gene transfer by viral vectors, e.g., retroviral vectors and adenoviral vectors, is higher than that of non-viral vectors. Retroviral vectors, including the most widely used amphotrophic murine leukemia virus (MuLV) vector, can infect only replicating cells, and typically, their transduction rate is lower than that of adenoviral vectors. However, since retroviral vectors integrate into the host genome the expression of the transgene is persistent. Recently retroviral vectors have been developed in which the therapeutic gene carrying vector construct is introduced into a packaging cell line that carries two independent constructs, which express structural proteins for packaging, thereby addressing safety issues surrounding the generation of replication competent retroviruses (Salmons and Gunzburg, 1997).

Adenoviral vectors can infect many cell types, resting and replicating, with high efficiency. Recently, a hybrid adeno/retroviral vector has been described. (See, e.g., Bilbao, et al., 1997.). Adeno-associated virus vectors also facilitate integration of transgenes into host chromosomes, and constitutive expression of a transgene, without evoking a strong host immune response.

Artificial chromosomes, e.g., yeast artificial chromosome (YAC) vectors may also be used to introduce heterologous nucleic acid constructs into cells.

Appropriate cloning and expression vectors for use in human cells are also described in Sambrook et al., 1989, and Ausubel F M et al., 1989, expressly incorporated by reference herein. The DNA coding sequence may be inserted into a plasmid or vector (collectively referred to herein as “vectors”) by a variety of procedures. In general, the DNA sequence is inserted into an appropriate restriction endonuclease site(s) by standard procedures. Such procedures and related sub-cloning procedures are deemed to be within the scope of knowledge of those skilled in the art.

Such vectors are typically equipped with selectable markers, insertion sites, and suitable control elements, such as termination sequences. The vector may comprise regulatory sequences, including, for example, non-coding sequences, such as introns and control elements, i.e., promoter and terminator elements or 5′ and/or 3′ untranslated regions, effective for expression of the coding sequence in host cells (and/or in a vector or host cell environment in which a modified soluble protein antigen coding sequence is not normally expressed), operably linked to the coding sequence. Large numbers of suitable vectors and promoters are known to those of skill in the art and many are commercially available.

Exemplary promoters include both constitutive promoters and inducible promoters, examples of which include a CMV promoter, an SV40 early promoter, an RSV promoter, an EF-1α promoter, a promoter containing the tet responsive element (TRE) in the tet-on or tet-off system as described (ClonTech and BASF), the beta actin promoter and the metallothienein promoter that can upregulated by addition of certain metal salts. Large numbers of suitable vectors and promoters are known to those of skill in the art, are commercially available and are described in Sambrook, et al., (supra). Hybrid promoters, which combine elements of more than one promoter also find utility in the methods of the invention. Methods for construction of hybrid promoters are well known in the art.

Selectable markers for use in such expression vectors are generally known in the art and the choice of the proper selectable marker will depend on the host cell. Typical selectable marker genes encode proteins that confer resistance to antibiotics or other toxins, for example, ampicillin, methotrexate, tetracycline, neomycin (Southern and Berg, J., 1982), mycophenolic acid (Mulligan and Berg, 1980), puromycin, zeomycin, or hygromycin (Sugden et al., 1985).

In one preferred embodiment of the invention, cytokine regulatory factor overexpression and/or the expression of an anti-apoptotic gene is achieved using cells that comprise exogenously provided cytokine regulatory factor and/or anti-apoptotic protein-encoding nucleic acid sequences, respectively, under the control of a suitable promoter, either constitutive or inducible, under conditions suitable for expression in cell culture.

B. Nucleic Acid Coding Sequences

A vector comprising a cytokine regulatory factor-encoding nucleic acid sequence may be introduced into a cell, resulting in overexpression of one or more cytokine regulatory factors by the cell.

Exemplary coding sequences for use in such vectors include, but are not limited to, the coding sequence from the human p68 PKR gene found at GenBank Accession No. M35663, the murine PKR gene and other eIF-2-alpha kinases including yeast GCN2 and hemin regulated inhibitor (Wek R C, Trends Biochem Sci 1994; 19: 491-496). Cytokine regulatory factors for use in practicing the invention include, but are not limited to, ISG (2-5A synthetase GenBank Accession No. NM_{—}006187, ISGγ3 GenBank Accession No. NM_{—}002038, MxA GenBank Accession No. 30817, MxB GenBank Accession No. 30818), IRF-1 GenBank Accession No. NM_{—}002198, IRF-3 GenBank Accession No. NM_{—}001571, IRF-7A GenBank Accession No. U53830, IRF-7B GenBank Accession No. U53831 IRF-7C GenBank Accession No. U53832, cytokine receptors, NF-kB GenBank Accession No. NM_{—}003998, AP-1 GenBank Accession No. J04111, NF-IL6 GenBank Accession No. X52560, PKC inducers, p38 MAPK GenBank Accession No. AF015256, Jak3 GenBank Accession No. NM_{—}000215, STAT GenBank Accession No. NM_{—}007315, IFN-γ GenBank Accession No. J00219, IFN-β GenBank Accession No. V00534, IFN-α GenBank Accession No. J00207, TNF-α GenBank Accession No. X02910, TNF-β GenBank Accession No. M16441, GM-CSF GenBank Accession No. NM_{—}00758, G-CSF GenBank Accession No. X03655, EGF GenBank Accession No. NM_{—}001963, PDGFalpha GenBank Accession No. NM_{—}002607, PDGFbeta GenBank Accession No. NM_{—}002609, TGFbeta GenBank Accession No. M60316, IL-1, chemokines (IL-8 GenBank Accession No. M28130, MIP-1a GenBank Accession No. NM_{—}002983, MIP-1b GenBank Accession No. J04130), monocyte chemotactic proteins (MCP1 GenBank Accession No. NM_{—}002982), PMA, calcium ionophores, sodium butyrate or endotoxin, polyl: C, dsRNA, viral analogs, cellular stress signals that activate PKR, (heat shock, pathogen infection), factors that interact with a promoter controlling PKR expression, signal of transduction and transcription (STAT) and any factor that effects enhanced cytokine production.

In addition, mutants or variant forms of a cytokine regulatory factor-encoding nucleic acid sequence can be included in vector constructs for use in the overexpression of the cytokine regulatory factor. The polynucleotides for use in practicing the invention include splice variants, sequences complementary to the native nucleic acid coding sequence and novel fragments thereof. The polynucleotides may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand. Upon expression, mutant or variant forms of these cytokine regulatory factors may have increased or decreased activity.

In one approach, mutant cytokine regulatory factor coding sequences are generated using the Transformer site-directed mutagenesis kit (ClonTech). Cai and Williams (J Biol Chem 1998; 273: 11274-11280) describe a series of PKR mutants that exhibit dissociation of substrate binding (in terms of eIF-alpha) from kinase activity (phosphorylation of substrates). These mutants which exhibit reduced PKR activity can be used for transfection of cells to generate PKR-expressing cells, albeit less effective in substrate binding or with less kinase activity.

A selected cytokine regulatory factor coding sequence may be inserted into a suitable vector according to well-known recombinant techniques and used to transform a cell line capable of cytokine regulatory factor overexpression.

In accordance with the present invention, polynucleotide sequences or genes which encode cytokine regulatory factors and anti-apoptotic proteins include splice variants, fragments of the full length genes, coding sequences for fusion proteins, modified forms of native or full length genes or functional equivalents thereof, collectively referred to herein as “cytokine regulatory factor-encoding nucleic acid sequences” and “anti-apoptotic protein-encoding nucleic acid sequences”, respectively.

Due to the inherent degeneracy of the genetic code, other nucleic acid sequences which encode substantially the same or a functionally equivalent amino acid sequence may be used to clone and express the CRF- or anti-apoptotic protein-encoding nucleic acid sequences. Thus, for a given CRF- or anti-apoptotic protein-encoding nucleic acid sequence, it is appreciated that as a result of the degeneracy of the genetic code, a number of coding sequences can be produced that encode the same amino acid sequence. For example, the triplet CGT encodes the amino acid arginine. Arginine is alternatively encoded by CGA, CGC, CGG, AGA, and AGG. Therefore it is appreciated that such substitutions in the coding region fall within the sequence variants covered by the present invention. Any and all of these sequence variants can be utilized in the same way as described herein for the native form of a CRF- or anti-apoptotic protein-encoding nucleic acid sequence.

A “variant” CRF- or anti-apoptotic protein-encoding nucleic acid sequence may encode a “variant” CRF- or anti-apoptotic amino acid sequence which is altered by one or more amino acids from the native polypeptide sequence, both of which are included within the scope of the invention. Similarly, the term “modified form of”, relative to a CRF- or anti-apoptotic protein, means a derivative or variant form of the native CRF- or anti-apoptotic protein-encoding nucleic acid sequence or the native CRF- or anti-apoptotic amino acid sequence. Typically, a “modified form of” a native CRF- or anti-apoptotic protein or the coding sequence for the protein has a derivative sequence containing at least one amino acid or nucleic acid substitution, deletion or insertion, respectively.

The polynucleotides for use in practicing the invention include sequences which encode native CRF- or anti-apoptotic proteins and splice variants thereof, sequences complementary to the coding sequence and novel fragments of CRF- or anti-apoptotic protein encoding polynucleotides. The polynucleotides may be in the form of RNA or DNA, and include messenger RNA, synthetic RNA and DNA, cDNA and genomic DNA. The DNA may be double-stranded or single-stranded and if single-stranded may be the coding strand or the non-coding (antisense, complementary) strand.

As will be understood by those of skill in the art, in some cases it may be advantageous to produce nucleotide sequences possessing non-naturally occurring codons. Codons preferred by a particular eukaryotic host (Murray, E. et al., 1989) can be selected, for example, to increase the rate of CRF- or anti-apoptotic protein expression or to produce recombinant RNA transcripts having desirable properties, such as a longer half-life than transcripts produced using the naturally occurring sequence.

A native CRF- or anti-apoptotic protein-encoding nucleotide sequence may be engineered in order to alter the coding sequence for a variety of reasons, including but not limited to, alterations which modify the cloning, processing and/or expression by a cell.

In one approach, a heterologous nucleic acid construct or expression vector for use in practicing the invention includes the coding sequence for a protein, the active form of which is desired such as the coding sequence for a cytokine regulatory factor (CRF), exemplified herein by PKR or the coding sequence for an anti-apoptotic protein, exemplified herein by CrmA, Bcl-2 or Bcl-X_{L}.

In one general embodiment of the invention, a CRF encoding nucleic acid sequence has at least 70%, preferably 80%, 85%, 90% or 95% or more sequence identity to the native coding sequence. For example, a coding sequence useful for expression of human PKR has at least 70%, preferably 80%, 85%, 90% or 95% or more sequence identity to the sequence found at GenBank Accession No. M35663.

In the case of a cytokine regulatory factor encoding nucleic acid sequences, the substitution, insertion or deletion may occur at any residue within the sequence, as long as the encoded amino acid sequence maintains the biological activity of the native cytokine regulatory factor.

In another general embodiment, the invention provides the nucleic acid coding sequence for CrmA, Bcl-2a, Bcl-X_{L} or a homologue thereof which has at least 70%, preferably 80%, 85%, 90% or 95% or more sequence identity to the native coding sequence found in GenBank. For example, a coding sequence useful for expression of the human p68 PKR gene has at least 70%, preferably 80%, 85%, 90% or 95% or more sequence identity to the sequence found at GenBank Accession No. M35663.

When introducing a variant form of the nucleic acid coding sequence for CrmA, Bcl-2a, Bcl-X_{L} or a homologue thereof into a human cell, the substitution, insertion or deletion may occur at any residue within the sequence, so long as the encoded amino acid sequence maintains the biological activity of the native anti-apoptotic protein.

In a related embodiment, apoptosis may be inhibited by the introduction of a modified coding sequence for a protein associated with apoptosis, e.g., eIF-2a or eIF-2alpha, eIF-3, FADD, Bcl-X_{S}, BAK, BAX, and the like. In this embodiment, the modified eIF-2a or eIF-2alpha, eIF-3, FADD, Bcl-X_{S}, BAK or BAX coding sequence encodes a derivative or variant form of the native protein that generally has a derivative polypeptide sequence containing at least one amino acid substitution, deletion or insertion, which is introduced at any residue within the polypeptide sequence such that it interferes with the biological activity of the protein.

Exemplary computer programs which can be used to determine identity between two sequences and thereby analyze variant coding sequences, include, but are not limited to, the suite of BLAST programs, e.g., BLASTN, BLASTX, and TBLASTX, BLASTP and TBLASTN, publicly available on the Internet on the NIH website. See, also, Altschul, S. F. et al., 1990 and Altschul, S. F. et al., 1997. Sequence searches are typically carried out using the BLASTN program when comparing a nucleic acid sequence relative to nucleic acid sequences in the GenBank DNA Sequences and other public databases. The BLASTX program is preferred for searching nucleic acid sequences which have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. [See, Altschul, et al., 1997.]

The degree of identity between two or more sequences in the analysis of variant coding sequences is generally performed using a sequence analysis program such as the CLUSTAL-W program (Thompson, J. D. et al., Nucleic Acids Research, 22:46734680. 1994) in MacVector version 6.5, operated with default parameters, including an open gap penalty of 10.0, an extended gap penalty of 0.1, and a BLOSUM 30 similarity matrix. Each of these sequence analysis programs is available at various locations on the Internet.

The relationship between two sequences may also be characterized by hybridization. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. “Maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity; while high stringency conditions are used to identify sequences having about 80% or more sequence identity.

Moderate and high stringency hybridization conditions are well known in the art (see, e.g., Sambrook, et al, 1989, Chapters 9 and 11, and in Ausubel, F. M., et al., 1993, expressly incorporated by reference herein). An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C.

The nucleic acid coding sequence for a cytokine regulatory factor may be altered for a variety of reasons, including but not limited to, alterations which modify the cloning, processing and/or expression of the cytokine regulatory factor by a cell.

Heterologous nucleic acid constructs may include the coding sequence for one or more cytokine regulatory factors alone or in combination with the coding sequence for one or more anti-apoptotic proteins, a variant, fragment or splice variant thereof: (i) in isolation; (ii) in combination with additional coding sequences; such as sequences encoding a fusion protein or signal peptide; (iii) in combination with non-coding sequences, such as introns and control elements, such as promoter and terminator elements or 5′ and/or 3′ untranslated regions, effective for expression of the coding sequence in a suitable host; and/or (iv) in a vector or host environment in which the coding sequence is a heterologous gene.

The present invention also makes use of recombinant nucleic acid constructs comprising one or more of the cytokine regulatory factor-encoding nucleic acid sequences alone or in combination with the coding sequence for one or more anti-apoptotic proteins, as described above. The constructs typically take the form of a vector, such as a plasmid or viral vector, into which the coding sequence has been inserted, in a forward or reverse orientation.

C. Selection and Transformation of Host Cells

A vector comprising a nucleic acid coding sequence, as described above, together with appropriate promoter and control sequences, is employed to transform a human cell to permit the cell to overexpress one or more cytokine regulatory factors alone or in combination with the coding sequence for one or more anti-apoptotic proteins and thereby enhance the production of mixtures of cytokines.

In one aspect of the present invention, a heterologous nucleic acid construct is employed to transfer the nucleic acid coding sequence into a cell in vitro, with established cell lines preferred. For long-term, high-yield production of mixtures of cytokines, stable expression is also preferred. It follows that any method effective to generate stable transformants may be used in practicing the invention.

The practice of the present invention will employ, unless otherwise indicated, conventional techniques of molecular biology, microbiology, recombinant DNA, and immunology, which are within the skill of the art. Such techniques are explained fully in the literature. See, for example, “Molecular Cloning: A Laboratory Manual”, Second Edition (Sambrook, Fritsch & Maniatis, 1989), “Animal Cell Culture” (R. I. Freshney, ed., 1987); “Current Protocols in Molecular Biology” (F. M. Ausubel et al., eds., 1987); and “Current Protocols in Immunology” (J. E. Coligan et al., eds., 1991), hereby expressly incorporated herein by reference.

A parental cell or cell line may be genetically modified (i.e., transduced, transformed or transfected) with a cloning vector or an expression vector. The vector may be in the form of a plasmid, a viral particle, a phage, etc.

Numerous methods for introducing nucleic acids into cells for expression of heterologous nucleic acid sequences are also known to the ordinarily skilled artisan, including, but not limited to electroporation; cell-to-cell fusion; nuclear microinjection or direct microinjection into single cells; bacterial protoplast fusion with intact cells; use of polycations, e.g., polybrene or polyomithine; membrane fusion with liposomes, lipofectamine or lipofection-mediated transfection; high velocity bombardment with DNA-coated microprojectiles; incubation with calcium phosphate-DNA precipitate; DEAE-Dextran mediated transfection; infection with modified viral nucleic acids; and the like. (See, e.g., Davis, L., Dibner, M., and Battey, I. Basic Methods in Molecular Biology, 1986.)

The genetically modified cells are cultured in conventional nutrient media modified as appropriate for activating promoters, selecting transformants or amplifying expression of the one or more nucleic acid coding sequences which were introduced into the cells. The culture conditions, such as temperature, pH and the like, are those previously used for the host cell selected for expression, and will be apparent to those skilled in the art. The progeny of cells into which one or more nucleic acid coding sequences have been introduced are generally considered to comprise the introduced sequence(s).

D. Enhancement of Cytokine Requlatory Factor and Cytokine Expression

In general, once a cell line that overexpresses one or more cytokine regulatory factors alone or in combination with one or more anti-apoptotic proteins has been generated, additional steps are taken to enhance production of mixtures of cytokines, and/or to facilitate recovery of cytokines from the cell culture. Such steps include one or more of (1) culturing the cells under conditions effective to enhance expression of one or more cytokine regulatory factors; (2) culturing the cells under conditions effective to facilitate recovery of cytokines; (3) priming the cells; and (4) treating the cells to induce production of one or more cytokine regulatory factors and one or more cytokines (induction).

Culturing the cells under conditions effective to enhance expression of one or more cytokine regulatory factors includes culture in medium containing factors that facilitate expression, e.g., a metal salt or antibiotics, added to the medium at a concentration effective to activate an inducible promoter.

Culturing the cells under conditions effective to facilitate recovery of cytokines includes culture in serum and/or protein-free medium.

Priming is a well known phenomenon whereby exposure of cells to a priming agent results in enhanced production of one or more cytokines, typically followed by induction. Exemplary priming agents include, but are not limited to, phorbol myristate acetate (PMA) and other phorbol esters, calcium ionophores, interferon-α, interferon-γ, interferon-β, G-CSF, GM-CSF, PDGF, TGF, EGF or chemokines (IL-8, MCP or MIP), sodium butyrate, endotoxin, PHA, LPS and derivatives thereof, such as 3-deacyl LPS, viruses such as Newcastle Disease Virus, a kinase activator (e.g., protein activator of PKR, PACT), or a transcription activator (NF-KB, IRFs including IRF-3 and IRF-7). Suppression of a PKR inhibitor, p53, has also been demonstrated to result in enhanced PKR activity (Tan S L, Gale M J Jr, Katze M G, Mol Cell Biol 18(5):2431-43, 1998). Alternatively, deprivation of serum and growth factors such as IL-3 may be used to induce PKR activity in cells. Suitable priming agent concentrations may be found in the scientific literature.

By way of example, a concentration of PMA in the range 5-50 nM, typically about 10 nM, is suitable. It will be understood that the optimal priming agent concentration and combination of priming agent, inducing agent and conditions for such priming and induction of a particular type of cells for production of a specific cytokine mixture will vary. However, such conditions may be determined by one of skill in the art without extensive experimentation.

Induction or treatment refers to the addition of a microbial, (viral, bacterial, or fungal) inducer, an extract of a microbe capable of acting as an inducer (e.g., an endotoxin or bacterial cell wall containing extract), or a non-microbial inducer to the cell culture. Exemplary non-microbial inducers include, but are not limited to, double-stranded RNA (dsRNA) such as poly(I):poly(C) or poly r(I):poly r(C) (poly IC) or viral dsRNA such as Sendai virus RNA, small molecules, e.g., polyanions, heparin dextran sulfate, chondroitin sulfate and cytokines.

Exemplary methods of viral induction include, but are not limited to, (1) exposure to live virus (such as Sendai virus, encephalomyocarditis virus or Herpes simplex virus); (2) exposure to the aforementioned killed virus; or (3) exposure to isolated double-stranded viral RNA. In addition, cytokine induction may be produced or enhanced by adding particular cytokines known to stimulate cytokine production in certain cells. After addition of the inducing agent, cells are generally further incubated until desired levels of induced and secreted cytokines are obtained. Incubation at 37° C. for at least 12-48 hours, and up to 72-96 hours is generally sufficient.

The inducing agent is added in an amount effective and for a period of time to induce cytokine production, e.g., effective to obtain production of a mixture of cytokines in the culture medium, e.g., from about 0.001-100 μg/ml.

In one exemplary application of the method, cells are primed with IFN-beta for approximately 24 hours, followed by exposure to medium containing polyI:C and cycloheximide for approximately 5 hrs, with Actinomycin D added during the last hour.

In another exemplary application of the method, cells are primed with IFN-beta for approximately 24 hours, then induced by treatment with a viral inducer, e.g., Sendai Virus (SV) for approximately 1 hr, followed by exposure to medium containing polyI:C and cycloheximide for approximately 5 hrs, with Actinomycin D added during the last hour.

Example 1 describes production of a CrmA expressing cell line and superinduction or viral induction of the cells. Example 2 describes exemplary vectors, transfection methods and production of a cytokine regulatory factor overexpressing cell line that also expresses the anti-apoptotic protein, Bcl-X_{L}, suitable for use in practicing the invention. Example 3 describes cytokine production by an exemplary cytokine regulatory factor overexpressing cell line and a cytokine regulatory factor overexpressing cell line that also expresses an anti-apoptotic protein.

In one preferred approach, overexpression of a cytokine regulatory factor and production of one or more cytokines is effected by further treatment of the cells with DEAE Dextran. In another approach, cytokine regulatory factor expression may be enhanced by another regulatory factor which interacts with the promoter controlling the expression of the cytokine regulatory factor-encoding nucleic acid sequence. Expression of the endogenous PKR-encoding nucleic acid sequence may be modulated by regulatory factors including the interferon-inducible GAS elements, the IL-6 sensitive NF-IL6 and APRF elements and NF-κB elements. (See, e.g., Jagus R. et al., 1999 and Williams B R, 1999.)

Typically, at various time points following culture, priming and treatment (i.e. induction), the culture medium is tested for the presence or one or more selected cytokines. The presence of selected cytokines may be assayed by direct detection, e.g., with an antibody binding assay or indirectly by the effect of the culture medium on the activity of various cytokine-responsive cells, according to well known biological assays.

In one exemplary approach, the culturing step is carried out in medium containing serum, and the induction step is carried out in medium that is substantially serum and/or protein free. This approach provides the advantage that the final cell culture medium from which the cytokine mixture is obtained has a minimum of added serum proteins, and thus lends itself to simpler purification methods in order to obtain a cytokine composition suitable for administration to humans.

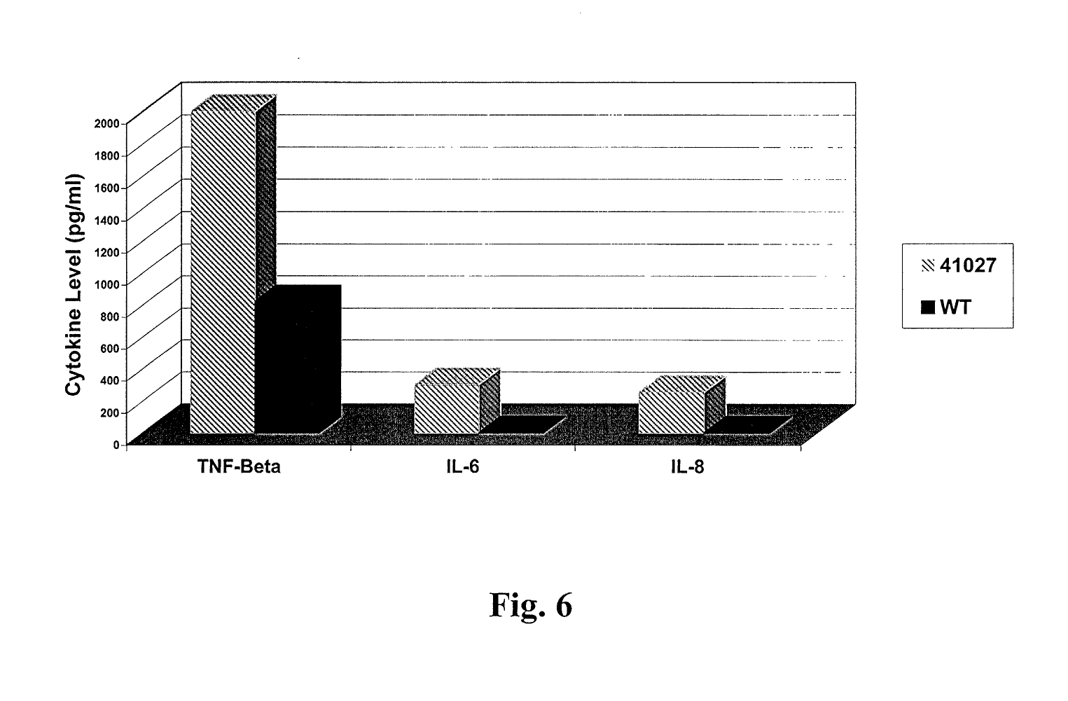

In another exemplary approach, priming of the PKR overexpressing Namalwa 41027 cell line with phorbol myristate acetate (PMA), followed by induction with polyI:C was effective to induce greater PKR expression than observed for wild type Namalwa cells. FIG. 6* illustrates enhanced TNF-beta, IL-6 and IL-8 production following poly I:C induction in Namalwa PKR+41027 cells relative to TNF-beta, IL-6 and IL-8 production by wild type Namalwa cells. These results demonstrate that a PKR overexpressing cell line derived by subcloning and selection produces a mixture of cytokines as further described in Example 3.

VII. Methods Of Evaluating Cytokine Regulatory Factor and Cytokine Expression

The activity, expression and/or production of cytokine regulatory factors and cytokines may be determined by methods known in the art. Examples include functional assays for biological activity and Northern blot or reverse transcriptase polymerase chain reaction (RT-PCR) for mRNA. Immunoassays, such as may be used to detect the expressed protein.

Alternatively, expression may be measured by immunological methods, such as immunohistochemical staining of cells or tissue sections to directly evaluate expression (e.g., indirect immunofluorescent assays), ELISA, competitive immunoassays, radioimmunoassays, Western blot, and the like. Antibodies useful for immunohistochemical staining and/or assay of sample fluids may be either monoclonal or polyclonal and may be prepared in any mammal.

By way of example, the presence, amplification and/or expression of an endogenous or exogenously provided PKR-encoding nucleic acid sequence may be measured in a sample directly, for example, by an assay for PKR activity, expression and/or production. Such assays include autophosphorylation assays, an assay for eIF2α phosphorylation, a kinase assay; Northern blotting to quantitate the transcription of mRNA, dot blotting (DNA or RNA analysis), RT-PCR (reverse transcriptase polymerase chain reaction), or in situ hybridization, using an appropriately labeled probe, based on the PKR-encoding nucleic acid sequence; and conventional Southern blotting.

The details of such methods are known to those of skill in the art and many reagents for practicing such methods are commercially available. In general, kits for cytokine analysis are commercially available and may be used for the quantitative immunoassay of the expression level of known cytokines or other proteins (e.g., cytokine detection kits available from R&D Systems).

VIII. Cytokine Production

Cytokines produced by the cells that overexpress a cytokine regulatory factor and/or an anti-apoptotic protein are secreted into the medium and may be purified or isolated, e.g., by removing unwanted components from the cell culture medium. In general, the cytokines are fractionated to segregate cytokines having selected properties, such as binding affinity to particular binding agents, e.g., antibodies or receptors; or which have a selected molecular weight range, or range of isoelectric points.

A. Purification and/or Isolation of Cytokines

After a combination of one or more of cell line selection, modification, priming and treating (i.e. induction) for an appropriate time period, production of a cytokine mixture is achieved. The culture medium containing the cell-produced cytokine mixture is then harvested and the cytokines are isolated and/or purified from the cell culture. To the extent that the harvested culture medium contains suspended cells, the medium may be centrifuged at low speed, filtered, or otherwise treated to remove cells and cellular debris. The medium may be further treated, e.g., by diafiltration or molecular sieve chromatography, to remove low molecular weight components, such as pyrogens, and higher molecular weight components that are outside the molecular weight range of cytokines, which is typically about 10-40 kD.

To obtain a desired cytokine mixture, the culture medium from cytokine-producing cells is subjected to various protein isolation procedures which take advantage of the binding affinity of each cytokine to binding agents such as antibodies or receptors, the molecular weight or isoelectric point thereof. Exemplary procedures include antibody-affinity column chromatography, ion exchange chromatography; ethanol precipitation; reverse phase HPLC; chromatography on silica or on a cation-exchange resin such as DEAE; chromatofocusing; SDS-PAGE; ammonium sulfate precipitation; or gel filtration using, for example, Sephadex G-75. Various methods of protein purification may be employed and such methods are known in the art and described for example in Deutscher, Methods in Enzymology, 182, 1990; Scopes, Protein Purification: Principles and Practice, Springer-Verlag, New York, 1982. The purification step(s) selected will depend on the nature of the production process used and the particular cytokine mixture produced.

In a preferred isolation method, a composition containing a desired cytokine mixture is co-purified by isolating the selected cytokines using affinity chromatography. The method employs, as the affinity medium, purified anti-cytokine antibodies or cytokine-specific receptors that bind to desired cytokines. Antibodies specific against a number of cytokines are commercially available, e.g., from Sigma Chemical Co., Sigma Catalog 2000-2001 (St. Louis, Mo.).

In addition, affinity columns suitable for cytokine-antibody interaction can be obtained from commercial vendors, e.g. Pharmacia. In using such columns to prepare a cytokine composition in accordance with the present invention, the column is equilibrated with solutions such as Tris-buffered saline (TBS) and selected antibodies to the desired cytokines are loaded on the pre-equilibrated affinity column to allow the antibodies to bind. The cytokine-containing culture medium, obtained directly from the cell culture or after partial purification is chilled to low temperature, e.g., 4° C., and loaded onto the affinity column. The column is then incubated on a tumbling device at room temperature for 12-18 hours to allow binding of the cytokines to the corresponding antibodies.

After incubation, the column is washed with one or more washing solutions, such as TBS, then the bound cytokines are then eluted with an appropriate eluting buffer. See, e.g., Sambrook J, et al., in MOLECULAR CLONING: A LABORATORY MANUAL, Cold Spring Harbor Laboratory Press, Cold Spring Harbor, N.Y., (1989) and U.S. Pat. Nos. 4,385,991, 3,983,001, 4,937,200, and 5,972,599, which provide details on the use of affinity chromatography for protein purification, each herein.

The chromatographic separation may be carried out by successively removing each individual cytokine from a selected cytokine mixture using each of a plurality of different affinity columns, and combining the resultant individually purified cytokines, or preferably, by mixing the cell medium with an affinity column material that contains a column plurality of attached antibodies against the several cytokines to be included in the final cytokine composition or mixture. According to another aspect of the invention, the cytokine mixture may be treated to remove cytokines that have little or no known value in the selected indication or which are known or likely to have a negative impact on therapeutic efficacy.

A cytokine composition or mixture of the present invention includes two or more cytokines produced by cytokine-producing cells prepared by the methods described herein. The cytokines in the mixture are selected for their particular biological activity alone or in combination with the other components of the mixture, and for their roles or potential benefit in treating cancer, viral infection, and inflammation. These determinations are made on the basis of existing clinical trial data, in vitro cellular studies and information from the scientific literature on the actions of various cytokines.

IX. Use of Cytokine Compositions of the Invention

The cytokine compositions or mixtures of the invention may be administered by routes including, but not limited to, oral delivery, intraocular delivery, transdermal delivery, transdermal (TD) injection, intramuscular (IM) injection, subcutaneous (SC) injection, intravenous (IV) injection, intraperitoneal (IP) injection, and peritumoral injection or by inhalation. The preferred route of administration is injection by intramuscular, subcutaneous or intravenous routes. Typically, treatment is continued until a desired endpoint is reached, for example, reduction in tumor load, clearing of viral infection, or lessening of symptoms of inflammation. As will be understood by those of skill in the art, the preferred indicators of treatment efficacy will vary dependent upon the disease condition under treatment. As with any therapeutic regimen, the duration of treatment with the cytokine mixtures of the invention will be adjusted according to the results of an evaluation of treatment efficacy. Such evaluations are conducted regularly, at a frequency appropriate to the disease condition under treatment. For example, an effective treatment of malignant cancers must prevent further spread of neoplastic cells and reduce mortality, i.e. increase survival time for patients who have the disease.

Suitable regimens for administration of cytokine mixtures are variable, but are typified by an initial administration followed by repeated doses at one or more intervals by subsequent administration.

In one exemplary application of the methods of the invention, the cytokine is interferon-alpha or interferon-beta and the dosage varies from about 3 to 5 million International Units to about 15 to 20 million International Units total cytokine concentration per administration per patient, where an average patient is 70 kg and a typical cytokine composition has a specific activity of from about 1×10⁸ IU per mg to 1×10⁹ IU per mg.

In another exemplary application of the methods of the invention, the cytokine is IL-2, GM-CSF or IFN gamma and the dosage varies from about 1 to 3 million International Units to about 5 to 10 million International Units total cytokine concentration per administration per patient, where an average patient is 70 kg.

In a typical therapeutic regimen, the cytokine mixture is administered 1 to 3 times per week for a period of 4 to 6 weeks. However, in some cases, administration of a cytokine mixture may be continued for an indefinite time of several years of more, e.g., in the case of administration of interferon-beta for the treatment of MS.

Surgery, radiation therapy, and chemotherapy are currently the primary methods for cancer treatment. It is contemplated that administration of a cytokine mixture may be combined with these other cancer therapies and new emerging treatment modalities including monoclonal antibodies and cancer vaccines. It will be appreciated that the methods described herein may interact in synergistic or additive ways with any one of surgery, radiation therapy, and chemotherapy, resulting in a greater therapeutic effect. In some cases, improved methods for treating cancer will combine conventional cancer treatments, erg. chemotherapy or radiation therapy, together with administration of a cytokine mixture.

The treatment regimens described above are presented for exemplary purposes and that the treatment regimen may be adjusted as needed, dependent upon the patient's response.

X. Exemplary Compositions and Methods

This section describes two exemplary compositions in accordance with the invention, and therapeutic applications of the two compositions.

A. Interferon γ.Compositions and Methods

In exemplary embodiments of the invention, the compositions include a mixture of interferon γ and at least one of human interferon α and interferon β in a mole ratio of between 1:1 to 1:1000 interferon γ to interferon α or human interferon β. In other exemplary embodiments, the composition includes human interferon γ and either or both of interferon α and human interferon β, at a mole ratio of between 1:10 to 1:100 interferon γ to interferon α and/or interferon β. The interferon α may include a mixture of interferon α subtypes and the mole ratio of human interferon γ to interferon α is calculated on the basis of the combined mole ratio of all of the subtypes present. More generally, the therapeutic compositions of the present invention may comprise mixtures of the type 2 human interferon, for example interferon γ, with at least one of the type 1 human interferons. Typically, the mole ratio of the type 1 interferon to type 2 interferon is in the range between 1:1 to 1:1000. More typically, the mole ration of the type 1 interferon to type 2 interferon is in the range between 1:10 to 1:100. In the case more than one type 1 interferons are included in the mixture, the mole ratio of the type 2 human interferon to type 1 interferon is calculated on the basis of the combined mole ratio of all the type 1 human interferons present.

Methods of producing interferons are generally known to the art and may includes production by human and genetically engineered non-human cells. Exemplary non-human cell lines which may be used for producing interferons used in the composition of the invention include E. coli and Chinese hamster ovary (CHO) cells. Preferably, the interferons of the present invention is derived from human cell lines. The most critical difference in proteins produced by human cells and by genetically engineered non-human cells is the status of glycosylation and sugar moiety structures. E. coli derived proteins are not glycosylated and CHO-derived proteins show different glycosylation patterns due to the lack of particular glycosyl transferases compared to human cells. These interferons may be prepared individually or as a mixture in a singly cell line. When produced individually, the interferons are mixed according to the preferred ratio.

A preferred method of producing a mixture of interferons has been described previously. Briefly, interferon-producing human cells are cultured in a culture medium, under conditions which stimulate interferon production. The desired mixtures, for example, interferon γ and interferon α, and interferon γ and interferon β, are then co-purified from the mixture and co-formulated.

Examples of parental cell lines for use in the production of mixtures of cytokines include, but are not limited to, cells of the monocyte/macrophage lineage, lymphocytic lineage cells including T- and B-cells, mast cells, fibroblasts, bone marrow cells, keratinocytes, osteoblast-derived cells, melanocytes, endothelial cells, platelets, various other immune system cells, lung epithelial cells, pancreatic parenchymal cells, glial cells and tumor cells derived from such cell types. The selection of a specific cell line for specific mixtures are generally known to those skilled in the art. Preferrably, the human cytokine-producing cells are T-cells, for example Jurkat an CEM cells. Also, preferably, the cell line use for interferon production comprising cells that have been selected, modified, primed and/or primed and induced in a manner effective to result in enhanced production of mixtures of cytokines relative to the corresponding parental cell line. Methods of priming and inducing the interferon-producing cells are described above.

In general, preferred culture conditions for a given cell line may be found in the scientific literature and/or from the source of the cell line such as the American Type Culture Collection. Preferred culture conditions for primary cell lines, such as fibroblasts, B-cells, T-cells, endothelial cells, dendritic cells, and monocytes are also generally available in the scientific literature. For example, the interferon producing cells may be cultured in a standard medium containing physiological salts and nutrients, such as standard cell culture media RPMI, MEM, IMEM, DMEM, or F12 which may be supplemented with 0.1-10% serum, such as fetal bovine serum. Alternatively, serum free andor animal-derived protein-free or protein-free medium may also be used.

Interferon mixtures are isolated and/or purified from the cell culture. The suspended cells can be separate from the growth medium, e.g., by centrifugation at low speed, filtered, or otherwise treated to remove cells and cellular debris. The medium may be further treated, e.g., by diafiltration or molecular sieve chromatography, to remove low molecular weight components, such as pyrogens, and higher molecular weight components that are outside the molecular weight range of cytokines, which is typically about 10-40 kD. The specific mixture may may isolated by various protein isolation procedures which take advantage of the binding affinity of the desired interferon to binding agents such as antibodies or receptors, the molecular weight or isoelectric point thereof. Exemplary procedures include antibody-affinity column chromatography, ion exchange chromatography; ethanol precipitation; reverse phase HPLC; chromatography on silica or on a cation-exchange resin such as DEAE; chromatofocusing; SDS-PAGE; ammonium sulfate precipitation; or gel filtration using, for example, Sephadex G-75. Various methods of protein purification may be employed and such methods are known in the art and described for example in Deutscher, Methods in Enzymology, 182, 1990; Scopes, Protein Purification: Principles and Practice, Springer-Verlag, New York, 1982.

In a preferred isolation method, a composition containing a desired cytokine mixture is co-purified by isolating the selected cytokines using affinity chromatography. The method employs, as the affinity medium, purified anti-cytokine antibodies or cytokine-specific receptors that bind to desired cytokines. Antibodies specific against a number of cytokines are commercially available, e.g., from Sigma Chemical Co., Sigma Catalog 2000-2001 (St. Louis, Mo.).

The composition of the invention may be formulated for a variety of uses and methods of administration including without limitation, injectable solutions, aerosols and dry powders. Typically, the compositions are formulated in a pharmaceutically liquid suitable for injection or as an aerosol suitable for inhalation.

For an injectable composition, the composition is either dried for later rehydration or suspended in a pharmaceutically acceptable liquid. Preferably, the liquid is an acceptable excipient such as water, saline, dextrose, glycerol and the like. The suspension can also contain small amounts of non-toxic auxiliary substances such as pH buffering agents, including sodium acetate, sodium phosphate, and the like. Typically, the composition as prepared can be administered intraveneously by injection.

For administration by inhalation, the interferon composition as produced may be reprocessed to enable aerosolization by an appropriate device. The reprocessing may simply be exchanging the suspending liquid with a suitable excipient which allows for the production of dropletts having mass median aerodynamic diameters in the range of 0.5 to 5 μm for nebulization. Suitable excipients include water, saline, dextrose, glycerol and the like.

In addition to nebulization, the interferon composition can be formulated into a dry powder suitable for aerosolization. The interferon composition may be dried first and then micronize to reduce the particle size or vice versa. Several conventional methods, which include freeze drying, spray drying, may be used to produce dry powders suitable for delivery to the lungs. Drying may also be accomplished by supercritical fluid processing.

The alpha-gamma interferon compositions of the invention is a broad spectrum anti-cancer agent and may be used for the treatment of cancers including but are not limited to non-Hodgkin's lymphoma, malignant melanoma, renal cell carcinoma, chronic myelogenous leukemia, multiple myeloma, hairy cell leukemia, AIDs-related Kaposi's sarcoma, basal cell carcinoma, metastatic breast cancer, cutaneous T-cell lymphoma, prostate cancer, colorectal cancer, liver cancer and mycosis fungoides. The beta-gamma interferon compositions of the invention are also anticancer agents and suitable for use to treat cancers, such as colorectal cancer, non-small-cell lung cancer, glioma, mycosis fungoides.

The alpha-gamma and beta-gamma interferon compositions of the invention may also be used for the treatment of viral infections including but are not limited to HIV, hepatitis C, hepatitis B, and other human-pathogenic viruses.

The cytokine compositions or mixtures of the invention may be administered by routes including, but not limited to, oral delivery, intraocular delivery, transdermal delivery, transdermal (TD) injection, intramuscular (IM) injection, subcutaneous (SC) injection, intravenous (IV) injection, intraperitoneal (IP) injection, and peritumoral injection or by inhalation. The preferred route of administration is injection by intramuscular, subcutaneous or intravenous routes. Typically, treatment is continued until a desired endpoint is reached, for example, reduction in tumor load, clearing of a viral infection. As will be understood by those of skill in the art, the preferred indicators of treatment efficacy will vary dependent upon the disease condition under treatment. As with any therapeutic regimen, the duration of treatment with the alpha and gamma interferon mixtures of the invention will be adjusted according to the results of an evaluation of treatment efficacy. Such evaluations are conducted regularly, at a frequency appropriate to the disease condition under treatment. For example, an effective treatment of malignant cancers must prevent further spread of neoplastic cells and reduce mortality, i.e. increase survival time for patients who have the disease.

Suitable regimens for administration of cytokine mixtures are variable, but are typified by an initial administration followed by repeated doses at one or more intervals by subsequent administration.

In one exemplary application of the methods of the invention, the interferon mixture is alpha-gamma or beta-gamma, the dosage of gamma should varies from 1 to 3 million IU to 5 to 10 million IU, with the total interferon concentration per administration, where an average patient is 70 kg and a typical interferon composition has a specific activity of from about 1×10⁸ IU per mg to 1×10⁹ IU per mg.

B. Interferon α and Interferon β.Compositions and Methods

In another general exemplary embodiment, the invention includes a composition of mixture of interferon α and human interferon β a mole ratio of between 1:10 to 10:1 human interferon α to human interferon β in a pharmaceutically acceptable carrier, or dried form. In other exemplary embodiments, the composition includes human interferon α and human interferon β, at a mole ratio of between 1:10 to 1:1. In another exemplary embodiments, the composition includes human interferon α and human interferon β, at a mole ratio of between 1:1 to 10:1. The interferon α may include a mixture of interferon α subtypes and the mole ratio of human interferon β to interferon α is calculated on the basis of the combined mole ratio of all of the subtypes present.

Methods of producing alpha and beta interferons are generally known to the art and may includes production by human and genetically engineered non-human cells. Exemplary non-human cell lines which may be used for producing interferons used in the composition of the invention include E. coli and Chinese hamster ovary (CHO) cells. Preferably, the interferons of the present invention is derived from human cell lines. The most critical difference in proteins produced by human cells and by genetically engineered non-human cells is the status of glycosylation and sugar moiety structures. E. coli derived proteins are not glycosylated and CHO-derived proteins show different glycosylation patterns due to the lack of particular glycosyl transferases compared to human cells. These interferons may be prepared individually or as a mixture in a singly cell line. When produced individually, the interferons are mixed according to the preferred ratio.

A preferred method of producing a mixture of interferons has been described previously. Briefly, interferon-producing human cells are cultured in a culture medium, under conditions which stimulate interferon production. The desired mixtures, for example, interferon γ and interferon α, and interferon γ and interferon β, are then co-purified from the mixture and co-formulated.

Examples of parental cell lines for use in the production of mixtures of cytokines include, but are not limited to, cells of the monocyte/macrophage lineage, lymphocytic lineage cells including T- and B-cells, mast cells, fibroblasts, bone marrow cells, keratinocytes, osteoblast-derived cells, melanocytes, endothelial cells, platelets, various other immune system cells, lung epithelial cells, pancreatic parenchymal cells, glial cells and tumor cells derived from such cell types. The selection of a specific cell line for specific mixtures are generally known to those skilled in the art. Preferrably, the human cytokine-producing cells are B-cells, for example Namalwa, 293 an Raji cells, where Namalwa cells are most preferred. Also, preferably, the cell line use for interferon alpha and beta production comprising cells that have been selected, modified, primed and/or primed and induced in a manner effective to result in enhanced production of mixtures of cytokines relative to the corresponding parental cell line. Methods of priming and inducing the interferon-producing cells are described above.

In general, preferred culture conditions for a given cell line may be found in the scientific literature and/or from the source of the cell line such as the American Type Culture Collection. Preferred culture conditions for primary cell lines, such as fibroblasts, B-cells, T-cells, endothelial cells, dendritic cells, and monocytes are also generally available in the scientific literature. For example, the interferon producing cells may be cultured in a standard medium containing physiological salts and nutrients, such as standard cell culture media RPMI, MEM, IMEM, DMEM, or F12 which may be supplemented with 0.1-10% serum, such as fetal bovine serum. Alternatively, serum free andor animal-derived protein-free or protein-free medium may also be used.

Interferon alpha-beta mixtures are isolated and/or purified from the cell culture. The suspended cells can be separate from the growth medium, e.g., by centrifugation at low speed, filtered, or otherwise treated to remove cells and cellular debris. The medium may be further treated, e.g., by diafiltration or molecular sieve chromatography, to remove low molecular weight components, such as pyrogens, and higher molecular weight components that are outside the molecular weight range of cytokines, which is typically about 10-40 kD. The specific mixture may may isolated by various protein isolation procedures which take advantage of the binding affinity of the desired interferon to binding agents such as antibodies or receptors, the molecular weight or isoelectric point thereof. Exemplary procedures include antibody-affinity column chromatography, ion exchange chromatography; ethanol precipitation; reverse phase HPLC; chromatography on silica or on a cation-exchange resin such as DEAE; chromatofocusing; SDS-PAGE; ammonium sulfate precipitation; or gel filtration using, for example, Sephadex G-75. Various methods of protein purification may be employed and such methods are known in the art and described for example in Deutscher, Methods in Enzymology, 182, 1990; Scopes, Protein Purification: Principles and Practice, Springer-Verlag, New York, 1982.

In a preferred isolation method, a composition containing a desired cytokine mixture is co-purified by isolating the selected cytokines using affinity chromatography. The method employs, as the affinity medium, purified anti-cytokine antibodies or cytokine-specific receptors that bind to desired cytokines. Antibodies specific against a number of cytokines are commercially available, e.g., from Sigma Chemical Co., Sigma Catalog 2000-2001 (St. Louis, Mo.).

The composition of the invention may be formulated for a variety of uses and methods of administration including without limitation, injectable solutions, aerosols and dry powders. Typically, the compositions are formulated in a pharmaceutically liquid suitable for injection or as an aerosol suitable for inhalation.

Generally, the composition first produced are suspended in a liquid. Preferably, the liquid is an acceptable excipient such as water, saline, dextrose, glycerol and the like. The suspension can also contain small amounts of non-toxic auxiliary substances such as pH buffering agents, including sodium acetate, sodium phosphate, and the like. Typically, the composition as prepared can be administered intraveneously by injection.

For administration by inhalation, the interferon composition as produced may be reprocessed to enable aerosolization by an appropriate device. The reprocessing may simply be exchanging the suspending liquid with a suitable excipient which allows for the production of dropletts having mass median aerodynamic diameters in the range of 0.5 to 5 μm for nebulization. Suitable excipients include water, saline, dextrose, glycerol and the like.

In addition to nebulization, the interferon composition can be formulated into a dry powder suitable for aerosolization. The interferon composition may be dried first and then micronize to reduce the particle size or vice versa. Several conventional methods, which include freeze drying, spray drying, may be used to produce dry powders suitable for delivery to the lungs. Drying may also be accomplished by supercritical fluid processing.

The alpha-beta interferon compositions of the invention is a broad spectrum anti-cancer agent and may be used for the treatment of cancers including but are not limited to non-Hodgkin's lymphoma, malignant melanoma, renal cell carcinoma, chronic myelogenous leukemia, multiple myeloma, hairy cell leukemia, AIDs-related Kaposi's sarcoma, basal cell carcinoma, metastatic breast cancer, cutaneous T-cell lymphoma, prostate cancer, colorectal cancer, liver cancer and mycosis fungoides.

The alpha-beta interferon compositions of the invention may also be used for the treatment of viral infections including but are not limited to HIV, hepatitis C, hepatitis B, and other human-pathogenic viruses.

The alpha-beta interferon compositions of the invention may also be used for the treatment of multiple sclerosis.

The alpha-beta compositions or mixtures of the invention may be administered by routes including, but not limited to, oral delivery, intraocular delivery, transdermal delivery, transdermal (TD) injection, intramuscular (IM) injection, subcutaneous (SC) injection, intravenous (IV) injection, intraperitoneal (IP) injection, and peritumoral injection or by inhalation. The preferred route of administration is injection by intramuscular, subcutaneous or intravenous routes. Typically, treatment is continued until a desired endpoint is reached, for example, reduction in tumor load, clearing of a viral infection and reduction of the inflammation. As will be understood by those of skill in the art, the preferred indicators of treatment efficacy will vary dependent upon the disease condition under treatment. As with any therapeutic regimen, the duration of treatment with the alpha-beta interferon mixtures of the invention will be adjusted according to the results of an evaluation of treatment efficacy. Such evaluations are conducted regularly, at a frequency appropriate to the disease condition under treatment. For example, an effective treatment of malignant cancers must prevent further spread of neoplastic cells and reduce mortality, i.e. increase survival time for patients who have the disease.

Suitable regimens for administration of cytokine mixtures are variable, but are typified by an initial administration followed by repeated doses at one or more intervals by subsequent administration.

In one exemplary application of the methods of the invention, the alpha-beta interferon mixture dosage varies from about 3 to 5 million International Units to about 15 to 20 million International Units total cytokine concentration per administration per patient, where an average patient is 70 kg and a typical interferon composition has a specific activity of from about 1×10⁸ IU per mg to 1×10⁹ IU per mg.

All patent and literature references cited in the present specification are hereby expressly incorporated by reference in their entirety.

The following examples illustrate but are not intended in any way to limit the invention.

### EXAMPLE 1

### Preparation and Characterization of a Transgenic CrmA-expressing Cell Line

A. Preparation of a Transgenic CrmA-Expressing Cell Line

The pEF FLAG-crmA-puro expression vector was constructed by inserting the coding sequence for CrmA in-frame into the pEF Bos vector described by Mizushima and Negata (NAR 18, 5322, 1990), based on the vector described by Huang et al., 1997. pEF FLAG-crmA-puro contains a full length cDNA encoding the anti-apoptotic CrmA protein (GenBank Accession No. M14217; Cowpox virus white-pock variant (CPV-W2) (CrmA) gene, complete coding sequence) under the control of the strong elongation factor 1 alpha (EF-1 alpha) promoter and the puromycin resistance gene under the control of the pGK promoter. An additional feature of note is the coding sequence for the N-terminal FLAG epitope (Hopp et al., 1988) that was added to the CrmA nucleic acid sequence to facilitate detection of cell lines that express CrmA.

The vector also includes (i) a polyadenylation signal and transcription termination sequence to enhance mRNA stability; (ii) an SV40 origin for episomal replication and simple vector rescue; (iii) an ampicillin resistance gene and a ColE1 origin for selection and maintenance in E. coli; and (iv) a puromycin resistance marker (Puro) to allow for selection and identification of plasmid-containing eukaryotic cells after transfection with pEF FLAG-crmA-puro.

The day before transfection MG-63 cells were seeded in a 6 well plate at 5×10⁴ per well. 2 μg of pEF FLAG-crmA-puro plasmid DNA was suspended in 100 μl Opti-MEM medium lacking serum, proteins or antibiotics. Lipofectamine reagent (Gibco, 10 μl) was diluted to 100 μl with Opti-MEM serum-free medium. Following gentle mixing of the two solutions, the mixture was incubated at room temperature for 45 min to allow for DNA-liposome complex formation. Immediately prior to treatment of MG-63 cells, 600 μl of Opti-MEM serum-free medium was added to the reaction tube containing the DNA-liposome mixture to obtain the final transfection solution. The cells were washed with PBS and followed by addition of the final DNA-liposome mixture and incubation for 4 hours at 37° C. This was followed by the addition of 1 ml MEM-5% FBS and incubation for an additional 16 hrs. The culture supernatant was removed by gentle aspiration and fresh cell growth medium (MEM supplemented with 5% FBS) added. After incubation for 48 hr, fresh media (MEM with 5% FBS) containing the selection marker, geneticin (G418, 500 μg/ml), was added to select for stable transfectants using standard methodology known in the art. In summary, a bulk population of stable transformants was obtained by selection with 500 μg/ml G418 (Gibco-BRL) for 3-4 weeks.

B. Characterization of a Transgenic CrmA-Expressing Cell Line

1. Increased Cell Viability

Wild type (WT) and CrmA-expressing (CrmA-#2) MG-63 cells were treated by Sendai virus (SV) induction and superinduction (SI; Inoue I et al., 1991) using the following procedure.

Cells were seeded at a cell density 2.5×10⁴ cells per well in 24 well plates, followed by incubation at 37° C. with CO₂ concentration at 5%. Following incubation, cells were primed with IFN-beta (100 lU/ml) for 24 hr. The cells were then induced by the addition of 1000 hemagglutinin units of SV in 200 μl of MEM medium supplemented with 2% fetal bovine serum (FBS) to each well, and incubation for one hour, followed by the addition of 300 μl of fresh medium containing polyI:C (100 μg/ml) and cycloheximide (5 μg/ml) and incubation for an additional 5 hrs. Actinomycin D was added during the last hour to a final concentration 4 μg/ml. After the induction process, the treated cells were washed 3 times with PBS to remove all inducers and resuspended in fresh MEM containing 2% FBS.

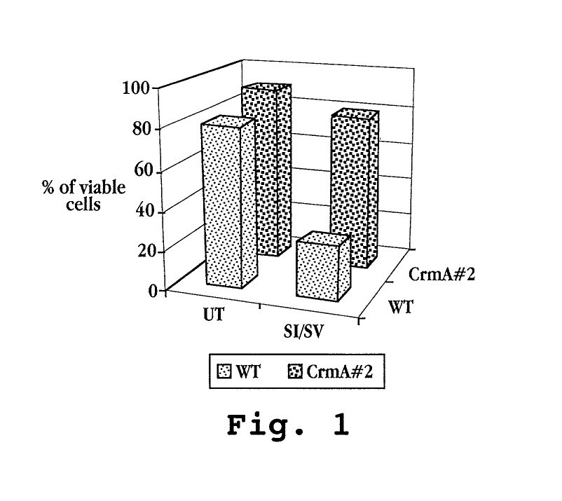

Wild type (WT) and CrmA-expressing (CrmA-#2) MG-63 cells that were not treated by Sendai virus (SV)± or superinduction (SI) were used as controls (UT). The viability of each type of cells was measured using a standard propidium iodide FACS assay. As shown in FIG. 1, CrmA expression inhibits SV/SI-induced cell death, indicated by a viability of up to 80% for CrmA-expressing cells at 20 h after SV induction and SI treatment. In contrast, only 20% of wild type MG-63 cells exposed to the same conditions survived the process.

2. Enhanced Production of Cytokines

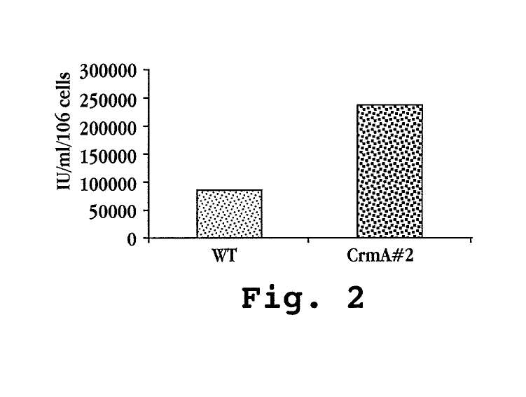

The cells were incubated for 20 hrs, then the culture medium from each well was collected and assayed for Interferon-beta (IFN-beta) production by ELISA. The IFN-beta ELISA was performed as described by the supplier. (Human Interferon-beta ELISA kit; distributed by TFB, Inc., and manufactured by FUJIREBIO, Inc., Tokyo, Japan). As shown in FIG. 2, there was significantly more IFN-beta produced by the CrmA#2 MG-63 cells, as compared to the MG-63 wild type counterparts.

CrmA-expressing (CrmA-#2) MG-63 cells were subjected to superinduction (SI) treatment in medium containing 0, 2 mM, 4 mM, and 8 mM of the nucleoside analog 2-aminopurine (2-AP), a known inhibitor of PKR.

SI (superinduction) treatment was carried out by seeding cells at a density of 2.5×10⁴ cells per well in 24 well plates at 37° C. at a CO₂ concentration of 5% the day before priming. Following incubation, the cells were primed with IFN-beta (100 IU/ml) for 24 hr, then 500 μl of fresh medium containing polyI:C (100 μg/ml) and cycloheximide (5 μg/ml) was added and the cells were incubated for an additional 5 hrs, with Actinomycin D added during the last hour to a final concentration 4 μg/ml. After the induction process, the treated cells were washed 3 times with PBS to remove all inducers and resuspended in fresh MEM containing 2% FBS.

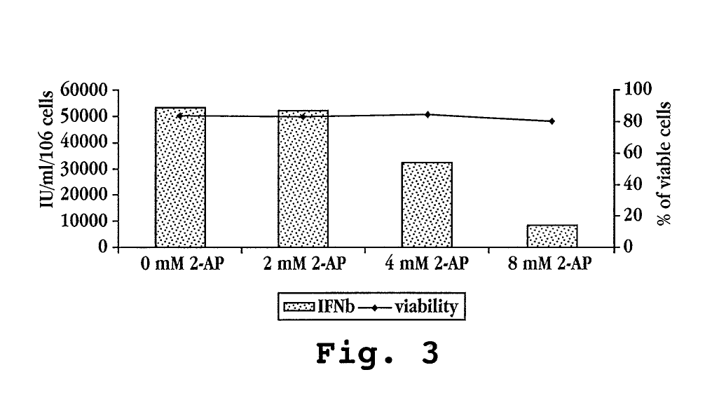

As shown in FIG. 3, 2-AP inhibited IFN-beta production in a dose-dependent manner, confirming that PKR plays a role in regulating IFN-beta expression.

3. Analysis of Flag-CrmA Protein Expression by Western Blot

Cells of the parental wild type cell line (MG-63-WT) and CrmA transformants (MG-63-CrmA-#2) prepared as set forth above, were cultured to 100% confluence in 100 mm dishes. Cells were washed in cold phosphate buffered saline (PBS) and collected in a 1.5 ml microcentrifuge tubes using a cell scraper. Following further washings with PBS, the cells were incubated in lysis buffer (10 mM Tris-HCL [pH 7.5], 1% Triton X-100, 0.25% SDS, 50 mM KCL, 1 mM dithiothreitol, 2 mM MgCl₂ and 1×Protein inhibitors cocktail [Roche]) for 10 min on ice, then centrifuged at 10,000 g for 10 min. The lysate supernatant was transferred to a new microcentrifuge tube and the protein concentration measured using a BRL kit following the protocol provided by the manufacturer. Cell lysates containing 100 μg of protein were loaded on a 4-12% NuPAGE Bis-Tris MOPS gel and subjected to electrophoretic separation, after which the gel was blotted onto a PVDF membrane. The membrane was further blotted in 5% milk-PBS overnight and exposed to primary rat anti-Flag antibodies, kindly provided by Dr. A Strasser (Royal Melbourne Hospital, Victoria, Australia) at dilutions of 1:500 for 1 hour. The blotted membrane was washed 3 times with PBS-0.1% Tween-20 and incubated with secondary anti-rat-HRP-conjugated antibodies (1:2000) for 1 hour. The presence of the Flag-CrmA protein was detected using ECL detection reagents (Amersham).

Each sample of cells transfected with a CrmA expression plasmid showed high levels of Flag-CrmA expression, in contrast to parental wild type control cells (MG63-WT) which showed no expression.

### EXAMPLE 2

### A Namalwa Cell Line that Overexpresses PKR Alone or PKR and an Anti-Apoptotic Protein

A. Preparation of pEF-FLAG-Bcl-X_{L}

The pEF-FLAG-Bcl-X_{L} vector (Huang, et al., 1997) contains a full length cDNA encoding the anti-apoptotic Bcl-X_{L} protein operably linked to the strong elongation factor 1 alpha (EF-1 alpha) promoter. An additional salient feature of the vector is the N-terminal FLAG epitope (Hopp et al., 1988) that was added to the Bcl-X_{L} protein to facilitate selection of cell lines that express high levels of Bcl-X_{L}.

The vector also includes i) a polyadenylation signal and transcription termination sequence to enhance mRNA stability; ii) a SV40 origin for episomal replication and simple vector rescue; iii) an ampicillin resistance gene and a ColE1 origin for selection and maintenance in E. coli; and iv) a puromycin resistance marker (Puro) to allow for selection and identification of the plasmid-containing eukaryotic cells after transfection of a Bcl-X_{L} and PKR.

B. Preparation of PcDNA-FLAG-PKR

The pcDNA-FLAG-PKR vector contains cDNA encoding the full-length human PKR molecule (551 amino acids; Meurs, et al., 1990; GenBank Accession No. NM002759) modified by the polymerase chain reaction to include the N terminal FLAG tag (Hopp et al., 1988) encoding the sequence MDYKDDDDK, and inserted into the eukaryotic expression vector pcDNA3 (Invitrogen), such that the FLAG-PKR coding sequence was expressed under the control of the CMV promoter.

The vector, termed pcDNA-FLAG-PKR, contains various features suitable for PKR transcription, including: i) a promoter sequence from the immediate early gene of the human CMV for high level mRNA expression; ii) a polyadenylation signal and transcription termination sequence from the bovine growth hormone (BGH) gene to enhance mRNA stability; iii) a SV40 origin for episomal replication and simple vector rescue; iv) an ampicillin resistance gene and a ColE1 origin for selection and maintenance in E. coli; and v) a G418 resistance marker (Neo) to allow for selection and identification of the plasmid-containing eukaryotic cells after transfection.

A second PKR vector, designated pTRE-PKR, was prepared by inserting the same PKR cDNA into a restriction/insertion site of a pTRE plasmid obtained from Clontech. The pTRE plasmid is similar to the pFLAG used in making the first-described PKR vector, but contains a tetracycline-responsive element upstream of the CMV promoter used to control the inserted gene.

C. Preparation of the 6A Cell Line (Bcl-X_{L} and PKR Positive)

The human B lymphoblastoid cell line Namalwa (WT) was transfected sequentially with the plasmids, pEF-FLAG-Bcl-X_{L} and pcDNA-FLAG-PKR. Stable transfectants were obtained by electroporation of 4×10⁶ exponentially growing Namalwa cells with 15 μg of the pEF-FLAG-Bcl-X_{L} plasmid in DMEM/F12 (+10% FBS) using a Gene Pulser apparatus (BioRad) set at 800 uF, 300V. Bulk populations of stable transformants were obtained by selection with 2 μg/ml puromycin (Gibco-BRL) for 3-4 weeks and screened for Bcl-X_{L} expression by flow cytometry as follows. The bulk transfectants were washed, permeabilized with acetone and subsequently stained with 2 μg/ml mouse anti-FLAG M2 monoclonal antibody (IBI) and then with phycoerythrin conjugated goat anti-mouse IgG (1 μg/ml; Becton-Dickinson). Cells were analyzed in the FACScan, live and dead cells being discriminated on the basis of their forward and side light-scattering properties and Bcl-X_{L} expressing cells by their level of fluorescence intensity. High level Bcl-X_{L} expressing transformants (Namalwa-Bcl-X_{L}) were then transfected with pcDNA-FLAG-PKR.

Stable high level Bcl-X_{L} expressing transfectants were obtained by electroporation of 4×10⁶ exponentially growing Namalwa-Bcl-X_{L} cells with 15 μg of the pcDNA-FLAG-PKR plasmid in DMEM/F12 (+10% FBS) using a Gene Pulser apparatus (BioRad) set at 800 μF, 300V. Bulk populations of stable transformants were obtained by selection with 2 mg/ml geneticin (G418, Gibco-BRL) for 3-4 weeks. Clonal lines were subsequently obtained by limiting dilution cloning and analyzed for Bcl-X_{L} and PKR expression by Western blot analysis (Huang et al., 1997). The proteins were identified using 2 μg/ml anti-FLAG M2 antibody followed by goat anti-mouse IgG-peroxidase conjugate and ECL detection (Amersham). An exemplary Bcl-X_{L} and PKR positive cell line was designated 6A.

D. Preparation of the A9 Cell Line (PKR Positive)

Stable high level PKR expressing transfectants were obtained by electroporation of 4×10⁶ exponentially growing Namalwa cells with 15 μg of the pTRE-PKR plasmid in DMEM/F12 (+10% FBS) using a Gene Pulser apparatus (BioRad) set at 800 μF, 300V. Bulk populations of stable transformants were obtained by selection with 2 mg/ml geneticin (G418, Gibco-BRL) for 3-4 weeks. Clonal lines were subsequently obtained by limiting dilution cloning and analyzed for PKR expression by Western blot analysis (Huang et al., 1997).

E. Characterization of a Transgenic Bcl-X_{L}- and PKR-Expressing Namalwa Cell Line

1. Increased Cell Viability

Wild type Namalwa cells (WT) and the A9 and 6A cells from Example 2C and 2D were examined for cell viability in culture under conditions of PKR overexpression and cytokine induction. Specifically, PKR and BcI-X_{L} double-transfected Namalwa cells (the 6A cell line), PKR-transfected Namalwa cells (the A9 cell line) and parental Namalwa cells (WT) were cultured at 2.5×10⁵ cells/ml in DMEM/F12 medium supplemented with 10% FBS. The cells were treated with 20 mM PMA (priming agent) for 20 hr followed by treatment with either 200 μg/ml poly r(I):poly r(C) and 10 μg/ml DEAE Dextran (poly IC induction) for 72 hr or 200 HAU/1×10⁶ cells of Sendai virus for 48 hr. Following treatment, cell viability was assessed by flow cytometry on a FACScan.

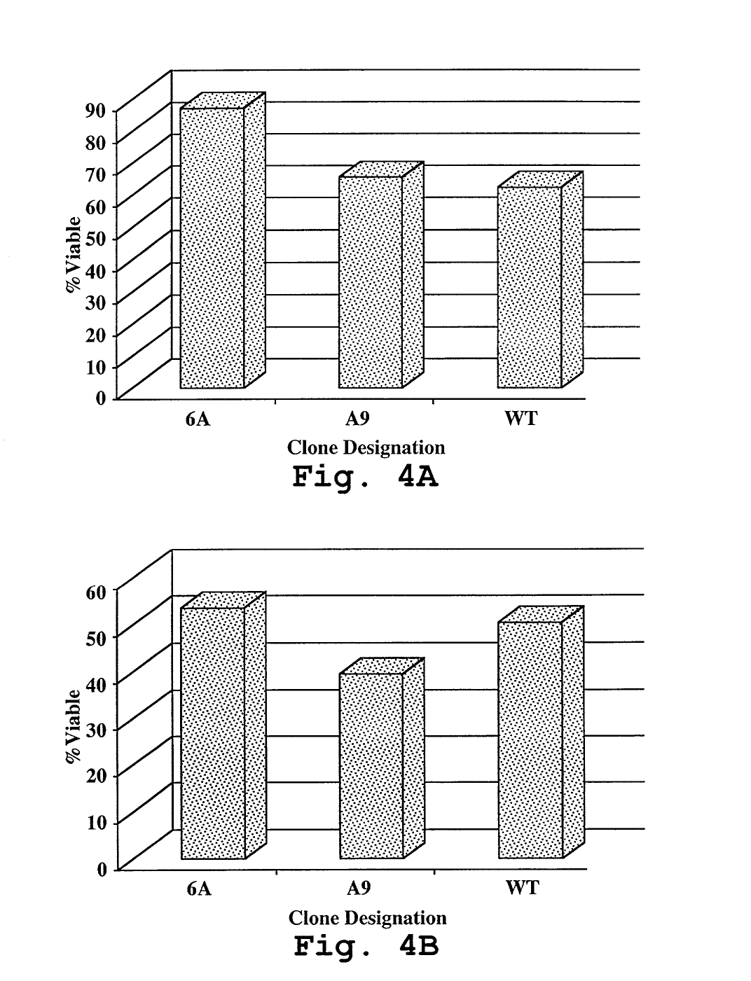

FIG. 4A shows that following Sendai virus induction, cell viability was similar for the PKR-transfected Namalwa cells (the A9 cell line) and parental Namalwa cells (WT), with greater viability observed for the PKR and Bcl-X_{L} double-transfected Namalwa cells (the 6A cell line).

FIG. 4B shows that following poly IC induction, cell viability was similar for the PKR and Bcl-X_{L} double-transfected Namalwa cells (the 6A cell line) and parental Namalwa cells (WT), with lower viability observed for PKR-transfected Namalwa cells (the A9 cell line).

2. Increased Expression of Interferon-α

The level of IFN-alpha production was also analyzed in the three cell lines following cytokine induction by poly IC and Sendai virus, both under conditions of PKR overproduction. The culture supernatants were collected and analyzed for IFN-alpha levels by ELISA according to the procedure provided by the supplier of the ELISA kits (R&D Systems).

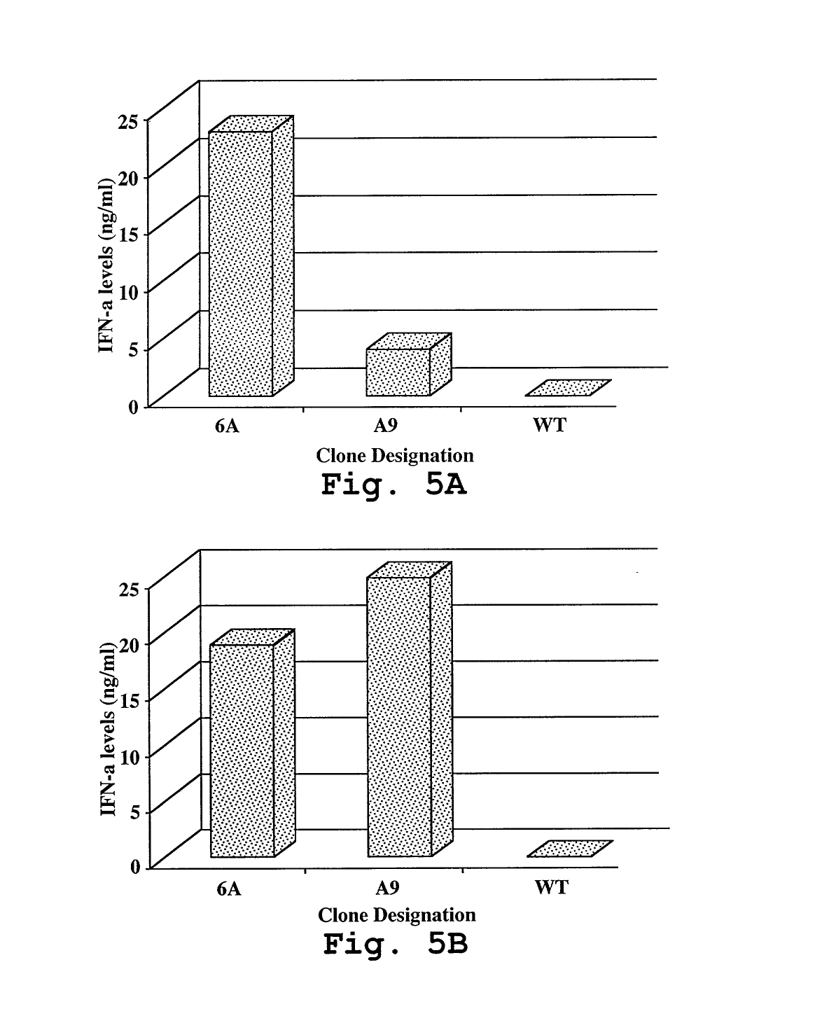

The results shown in FIG. 5A indicate that following Sendai virus induction, IFN-alpha production by PKR and Bcl-X_{L} double-transfected Namalwa cells (the 6A cell line) was significantly greater than IFN-alpha production by PKR-transfected Namalwa cells (the A9 cell line) and parental Namalwa cells (WT).

The results shown in FIG. 5B indicate that following poly IC induction, IFN-alpha production by PKR-transfected Namalwa cells (the A9 cell line) and PKR and Bcl-X_{L} double-transfected Namalwa cells (the 6A cell line) was significantly greater than IFN-alpha production by parental Namalwa cells (WT).

### EXAMPLE 3

### Preparation and Cytokine Production by PKR-Overexpressing Namalwa Cell Lines

A. Preparation of Cytokine- and Therapeutic Protein-Overexpressing Namalwa Cell Lines

Wild type Namalwa cells were obtained from the American Type Culture Collection (ATCC), 10801 University Blvd., Manassas, Va., U.S.A. These cells were cultured, subcloned, selected, primed and induced as detailed below.

Wild type Namalwa cells were cultured in DMEM/F12 medium containing fetal bovine serum with a range of concentrations of 0.5 to 15%. Individual cells were subjected to limiting dilution cloning (“subcloning”) by culturing in 96-well plates, using standard methods routinely employed by those of skill in the art. The subcloning step was carried out from 1 to 5 times and subclones were grown to obtain a population of approximately 0.3 to 0.5 million cells/ml using culture conditions typically employed to culture the parental cell line. Subclones were then assayed for PKR expression and activity by Northern blot, Western blot, PKR autophosphorylation assay (Der and Lau Proc Natl Acad Sci 92: 8841-8845, 1995), an assay for histone phosphorylation using methods published for measuring eIF2α phosphorylation (Zamanian-Daryoush, Der, Williams Oncogenes 18: 315-326, 1999), and a kinase assay (carried out by immunoprecipitation of PKR and in vitro assay for kinase activity (Zamanian-Daryoush, et al., Molecular and Cellular Biology, 20:1278-1290, 2000).

Subclones which exhibited at least 2-fold more kinase activity than the parental cell line were selected. Selected subclones were also screened for PKR production and/or expression by Western and Northern blot. Selected subclones were then induced to further enhance PKR and cytokine activity, production and/or expression, with or without priming prior to induction.

Exemplary PKR overexpressing cell lines, designated Namalwa PKR++41027 cells, subclone: 2A1.D1.G7.C1.A9, and Namalwa PKR++41027 cells, subclone: 2A1.D1.G7.G3.C1 are used herein to illustrate the invention.

B. Characterization of the PKR-Overexpressing Namalwa Cells

1. Expression of TNF-beta, IL-6 and IL-8 By Subclones of Namalwa PKR++41027 Cells

Namalwa PKR++41027 cells, subclone: 2A1.D1.G7.C1.A9 and WT Namalwa cells were pretreated with 20 nM phorbol myristate acetate at a cell density 5.0×10⁵ cells/ml, in 6 well plates for 20 hr. This was followed by treatment with 200 μg/ml polyI:C for an additional 72 hrs to induce cytokine expression. Culture supernatants from the treated cells were removed at 24 h, 48 h, and 72 h post induction and evaluated for TNF-beta, IL-6 and IL-8 production via ELISA (R & D Systems).

FIG. 6 illustrates enhanced TNF-beta, IL-6 and IL-8 production following poly I:C induction in Namalwa PKR++41027 cells, subclone: 2A1.D1.G7.C1.A9 relative to TNF-beta, IL-6 and IL-8 production by wild type Namalwa cells. The level of TNF-beta production was greatest at 72 hours post-induction, at which point the Namalwa PKR++41027 cells, subclone: 2A1.D1.G7.C1.A9 exhibited approximately 3 fold (3×) greater TNF-beta production than wild type Namalwa cells. Similarly at 72 hours post-induction with polyI:C, there was more than a 10-fold induction of IL-6 and IL-8 in the Namalwa PKR++41027 cells, subclone: 2A1.D1.G7.C1.A9 relative to that of the parental controls (FIG. 6).

2. Enhanced IFN-gamma Production in a Cytokine- and Therapeutic Protein-Over Expressing Cell Line

Namalwa PKR++41027 Cells, subclone: 2A1.D1.G7.G3.C1 were pretreated with 20 nM phorbol myristate acetate at a cell density 5.0×10⁵ cells/ml in 6-well plates at 37° C. for 20 hr to prime cytokine expression. This treatment was followed by exposure to 100 μg/ml poly(I)poly(C) plus 10 ug/ml DEAE dextran for an additional 48 hrs to induce cytokine expression. Culture supernatants from the treated cells were removed at 48 hr post induction and evaluated for IFN-gamma expression and secretion by ELISA using a kit obtained from R & D Systems. The ELISA was performed according to the manufacturers directions.

3. Enhanced Production of Multiple Cytokines in a Cytokine- and Therapeutic Protein-Over Expressing Cell Line

Namalwa PKR++41027 Cells, subclone: 2A1.D1.G7.C1.A9 cultured in DME/F12 medium supplemented with 10% FBS were pretreated with 1 mM sodium butyrate at a cell density of 7 to 8×10⁵ cells/ml in 6-well plates at 37° C. for 24 hr to prime cytokine expression. This treatment was followed by exposure to Sendai virus, 100 HA units for an additional 24 to 48 hr to induce cytokine expression. Culture supernatants from the treated cells were removed at 24 or 48 hr post induction and evaluated for cytokine or therapeutic protein expression and secretion by ELISA using kits obtained from R & D Systems according to the manufacturers directions. The results indicating the level of detected cytokines or therapeutic protein expression are provided in Table 2.

TABLE 2
Cytokine Or Other Therapeutic Protein
Expression from Namalwa 41:027 Cells^{&}

| Cytokine/Other | Level of Protein Expression¹ |
|---|---|
| Therapeutic Protein | Induced/uninduced cells |
| IFN-alpha | 180 ng/ml / Not done |
| IFN-beta | 267 IU/ml / <2.5 IU/ml |
| IFN-gamma | 1.6 ng/ml / <15 pg/ml |
| IL-1beta | <4 pg/ml / <4 pg/ml |
| IL-2 | <31 pg/ml / <31 pg/ml |
| IL-4 | 12 pg/ml / 8 pg/ml |
| IL-6 | 31 pg/ml / <3 pg/ml |
| IL-8 | 322 ng/ml / 54 ng/ml |
| IL-10 | 17 pg/ml / 5 pg/ml |
| GM-CSF | <8 pg/ml / <8 pg/ml |
| TNF-alpha | 328 pg/ml / 2 pg/ml |
| TNF-beta | 966 pg/ml / <31 pg/ml |
| Basic FGF | <10 pg/ml / <10 pg/ml |
| PDGF-BB | <31 pg/ml / <31 pg/ml |
| VEGF | 394 pg/ml / 677 pg/ml |

^{&}All proteins measured from Namalwa PKR++41027 cells, subclone: 2A1.D1.G7.C1.A9 except for IFN-gamma which was measured from Namalwa PKR++41027 Cells, subclone: 2A1.D1.G7.G3.C1.

### ¹EXAMPLE 4

Wild type (WT), CrmA or CrmA and PKR-expressing MG-63 cells were prepared and treated to induce cytokine production as further described below. ¹The level of cytokine expression detected under any particular set of priming and induction conditions is not representative of the full complement nor the absolute level of cytokines a given cell line is capable of expressing. Parameters including but not limited to, the choice and concentration of priming and induction agent(s), incubation temperature, the media and media additives, pH, cell density, culture vessel configuration, aeration, stirring and other culture conditions could affect the final level of cytokine expression.

A. MG-63 Cells Transfected With CrmA or CrmA and PKR Produce Mixtures of Cytokines

Human osteoblastoma MG-63 cells were obtained from ATCC and maintained in MEM supplemented with 5% FBS at 37° C. in the presence of 5% CO₂. THE MG-63 cells were transfected with (1) PEF Flag-CrmA (puromycin) plasmid (a gift from Dr. Strasser), for which a plasmid map and construction methods are described in Huang D C et al, (Oncogene Jan. 30, 1997;14(4):405-14), using Lipofectamine (using the procedures suggested by the (manufacturer; Gibco-BRL). Individual cell clones were isolated by limiting dilution (3-5 cells/ml) and selection of individual antibiotic-resistant colonies using 96 well plates.

CrmA-expressing MG-63 cells were subsequently transfected with the pet V5-PKRwt plasmid (blasticidin; provided by Dr. Williams B. R. G.; Cleveland Clinic Foundation), using Lipofectamine (Gibco-BRL) and stable cell lines were selected in the presence of 1.5 μg/ml puromycin and 10 μg/ml blasticidin (Invitrogen Corporation, Carlsbad, Calif.). Individual cell clones were isolated by limiting dilution (3-5 cells/ml) and selection of individual blasticidin-resistant colonies using 96-well plates. The expression of PKR was evaluated by Western blot using V5 epitope tagged antibodies (Invitrogen).

SI (superinduction) was performed as described by Inoue I et al., 1991. One day before superinduction, cells were seeded in 6-well plates and incubated at 37° C. in the presence of 5% CO₂. The next day, cells were primed with 100 IU/ml IFN-beta and 1 mM Na Butyrate. After 24 hrs., fresh medium containing polyI:C (100 μg/ml) and cycloheximide (5 μg/ml) was added for 5 hrs. Actinomycin D was added during the last hour to a final concentration of 4 μg/ml. Then the cells were washed 3 times with PBS to remove all inducers and the medium was changed to MEM containing 5% serum. After incubation for 20 hrs, the medium was collected for cytokine analysis by ELISA, with assays performed according to the manufacturer's instructions (R&D Systems and BioSource International). The results of the analyses are presented in Table 3 which shows that of the cytokines analyzed, MG-63 cells express IFN beta, IL-6, IL-8, GM-CSF, G-CSF, FGF and VEGF.

TABLE 3
Cytokine Or Other Therapeutic Protein Expression from MG-63 Cells

| Cytokine/Other | | Gene(s) Introduced |
|---|---|---|
| Therapeutic Protein | Level of Protein Expression | Into MG-63 Cells |
| IFN-beta | 125,000 IU/ml per 10⁶ cells | CrmA |
| | 160,000 IU/ml per 10⁶ cells | CrmA/PKR |
| IL-6 | 40 ng/ml per 10⁶ cells | CrmA |
| | 40 ng/ml per 10⁶ cells | CrmA/PKR |
| IL-8 | 340 ng/ml per 10⁶ cells | CrmA |
| | 622 ng/ml per 10⁶ ceIls | CrmA/PKR |
| GM-CSF | 340 pg/ml/10⁶ cells | CrmA |
| | 800 pg/ml/10⁶ cells | CrmA/PKR |
| G-CSF | 104 pg/ml | CrmA |
| | 117 pg/ml | CrmA/PKR |
| FGF | 13 pg/ml | CrmA |
| VEGF | 44 pg/ml | CrmA |

### EXAMPLE 5

Expression Analysis

The cytokine expression profile of Namalwa PKR++41027 cells, subclone: 2A1.D1.G7.C1.A9 was determined using an Affymetrix (Santa Clara, Calif.) human Gene chip as described in PNAS 1998, 95:15623-15628.

RNA was prepared for expression analysis from Namalwa PKR++41027 Cells, subclone: 2A1.D1.G7.C1.A9 cultured in DME/F12 medium supplemented with 10% FBS in roller bottles. The cells were primed with 1 mM sodium butyrate at a cell density of 7 to 8×10⁵ cells/ml at 37° C. for 24 hr., centrifuged at 1200 rpm and resuspended at a density of 1×10⁷ cells per ml in fresh growth medium with 1% FBS. This treatment was followed by exposure to Sendai virus, 100 HA units for an additional 60 to 90 min followed by the addition of a 3-fold volume of fresh medium and a 48 hr incubation to induce cytokine expression. Cells were harvested at the end of the treatment to prepare RNA for gene chip analysis by the single-step acid guanidinium thiocyanate/phenol/chloroform extraction method as described in Anal. Biochemistry 162, 156-159 (1987).

TABLE 4
Results of Gene Chip expression of A9
(PKR-transfected) Namalwa cells following induction

| Protein | Avg Diff | Description of Protein |
|---|---|---|
| Apoptosis- | 1,218 | Cluster including AF022385:Homo sapiens |
| related | | apoptosis-related protein TFAR15 (TFAR15) |
| protein | | mRNA |
| BMP 11 | 513.9 | Cluster including AF100907:Homo sapiens |
| | | bone morphogenetic protein 11 (BMP11) |
| | | mRNA |
| Caspase-like | 1,390 | AF005775 Homo sapiens caspase-like |
| apoptosis | | apoptosis regulatory protein 2 (clarp) |
| regulatory | | mRNA, alternatively spliced |
| protein 2 | | |
| CD27 | 18,475 | Cluster including M63928:Homo sapiens |
| | | T cell activation antigen (CD27) mRNA |
| CD27L | 17,166 | Cluster including L08096:Human CD27 |
| | | ligand mRNA |
| Chemokine | 3,006 | D43767=HUMAR Human mRNA for |
| | | chemokine |
| Chemokine | 611.5 | Cluster including U86358:Human chemokine |
| (TECK) | | (TECK) mRNA |
| Death domain | 795 | U84388=HSU84388 Human death domain |
| containing | | containing protein CRADD mRNA |
| prot CRADD | | |
| EGF | 813 | X04571=HSEGFRER Human mRNA for |
| | | kidney epidermal growth factor (EGF) |
| | | precursor |
| FGF 18 | 309 | Cluster including AF075292:Homo sapiens |
| | | fibroblast growth factor 18 (FGF18) mRNA |
| Gastrin- | 223 | K02054/FEATURE=mRNA/DEFINITION= |
| releasing | | HUMGRP5E Human gastrin-releasing |
| peptide | | peptide mRNA, complete cds |
| GH-1/GH-2/ | 9,416 | Cluster including J03071:Human growth |
| CS-1/CS-2 | | hormone (GH-1 and GH-2) and chorionic |
| | | somatomammotropin (CS-1, CS-2 and CS-5) |
| | | genes |
| Heat shock | 2,313 | Cluster including M64673:Human heat shock |
| factor 1 | | factor 1 (TCF5) mRNA |
| Heat shock | 1655.3 | M64673=HUMHSF1 Human heat shock |
| factor 1 | | factor 1 (TCF5) mRNA |
| Heat shock | 567 | Cluster including M65217:Human heat shock |
| factor 2 | | factor 2 (HSF2) mRNA |
| Hepatoma- | 2,910 | Cluster including D16431:Human mRNA for |
| derived | | hepatoma-derived growth factor |
| growth factor | | |
| HGF-like | 684 | Cluster including U28055:Homo sapiens |
| protein | | hepatocyte growth factor-like protein |
| | | homolog mRNA |
| HSP 70 | 21,120 | Heat Shock Protein, 70 Kda |
| HSP 70 | 11,072 | Heat Shock Protein, 70 Kda |
| HSP 70 | 858 | L12723=HUMHSP70H Human heat shock |
| | | protein 70 (hsp70) mRNA |
| HSP 70B | 508 | X51757/FEATURE=cds/DEFINITION= |
| | | HSP70B Human heat-shock protein HSP70B |
| | | gene |
| HSP 90 | 16,057 | Cluster including M16660:Human 90-kDa |
| | | heat-shock protein gene |
| HSP 90 | 13,458 | Cluster including X15183:Human mRNA for |
| | | 90-kDa heat-shock protein |
| HSP E | 2238.2 | Cluster including L08069:Human heat shock |
| | | protein, E. coli |
| IFN-a | 9,344 | M28585=HUMIFNN Human leukocyte |
| | | interferon-alpha mRNA |
| IFN-a | 8,053 | J00207=HUMIFNAA Human leukocyte |
| | | interferon (leif) alpha-a gene |
| IFN-a a | 2817.8 | J00207=HUMIFNAA Human leukocyte |
| | | interferon (leif) alpha-a gene |
| IFN-a | 2,019 | Cluster including V00541:Messenger RNA |
| | | for human leukocyte interferon |
| IFN-a d | 9,300 | J00210=HUMIFNAD Human leukocyte |
| | | interferon (IFN-alpha) alpha-d gene |
| IFN-a5 | 2,522 | X02956=HSIFNA5 Human interferon alpha |
| | | gene IFN-alpha 5 |
| IFN-a6 | 1345.6 | X02958=HSIFNA6 Human interferon alpha |
| | | gene IFN-alpha 6 |
| IFN-a-M1 | 6,154 | M27318=HUMIFNAM1 Human interferon |
| | | (IFN-alpha-M1) mRNA |
| IFN-b | 8,284 | V00535=HSIFD6 Gene for human fibroblast |
| | | interferon beta 1 |
| IFN-g | 6,263 | Cluster including L07633:Homo sapiens |
| | | (clone 1950.2) interferon-gamma IEF SSP |
| | | 5111 mRNA |
| IFN-g | 152 | Cluster including X13274:Human mRNA for |
| | | interferon IFN-gamma |
| IFN-omega | 2,356 | Cluster including X58822:Human |
| | | IFN-omega 1 gene for interferon-omega 1 |
| IGF-II | 1,217 | M13970=HUMGFI21 Human insulin-like |
| | | growth factor (IGF-II) gene, exon 1 of 4 |
| IL-1b | 291 | Cluster including M15330:Human |
| | | interleukin 1-beta (IL1B) |
| IL-1R2 | 1,522 | X59770=HSIL1R2II H. sapiens IL-1R2 |
| | | mRNA for type II interleukin-1 receptor |
| IL-1ra | 1,816 | Cluster including X52015:H.sapiens mRNA |
| | | for interleukin-1 receptor antagonist |
| IL-3 | 171 | M20137=HUMIL3A Human interleukin 3 |
| | | (IL-3) mRNA |
| IL-4 | 349 | M13982=HUMIL4 Human interleukin 4 |
| | | (IL-4) mRNA |
| Inhibitor of | 11,479 | U45878=HSU45878 Human inhibitor of |
| apoptosis | | apoptosis protein 1 mRNA |
| protein 1 | | |
| Lipocortin 2 | 13253.2 | Cluster including M62895:Human lipocortin |
| | | (LIP) 2 pseudogene mRNA |
| Lipocortin II | 11,053 | D00017=HUMLIC Homo sapiens mRNA for |
| | | lipocortin II |
| Macrophage- | 5,397 | Cluster including U83171:Human |
| derived | | macrophage-derived chemokine precursor |
| chemokine | | (MDC) mRNA |
| MIF | 21,100 | L19686=HUMMIF Homo sapiens |
| | | macrophage migration inhibitory factor |
| | | (MIF) gene |
| Monocyte- | 1,520 | Cluster including U49020:Human myocyte- |
| specific | | specific enhancer factor 2A (MEF2A) gene |
| enhancer factor | | |
| Myelin-assoc | 334 | Cluster including 028113:Human mRNA for |
| oligoden. | | MOBP (myelin-associated oligodendrocytic |
| basic prot | | basic protein) |
| Myocyte- | 1520.2 | Cluster including U49020:Human myocyte- |
| specific | | specific enhancer factor 2A (MEF2A) gene |
| enhancer factor | | |
| NK enhancing | 2,529 | Cluster including L19185:Human natural |
| factor | | killer cell enhancing factor (NKEFB) mRNA |
| Oral tumor | 4923.8 | Cluster including AF006484:Homo sapiens |
| suppressor | | putative oral tumor suppressor protein |
| protein (doc-1) | | (doc-1) mRNA |
| Osteogenic | 2356.6 | Cluster including X51801:Human OP-1 |
| protein | | mRNA for osteogenic protein |
| PK inhibitor | 158.4 | Cluster including S76965:protein kinase |
| | | inhibitor [human, neuroblastoma cell line |
| | | SH-SY-5Y |
| PK inhibitor | 846.1 | Cluster including AB019517:Homo sapiens |
| gamma | | PKIG mRNA for protein kinase inhibitor |
| | | gamma |
| PKCI-1 | 12,528 | U51004=HSU51004 Homo sapiens protein |
| | | kinase C inhibitor (PKCI-1) |
| Pre-B cell | 733 | Cluster including U02020:Human pre-B cell |
| enhancing | | enhancing factor (PBEF) mRNA |
| factor | | |
| Prothymosin | 5,279 | Cluster including M14630:Human |
| alpha | | prothymosin alpha mRNA |
| RANTES | 22,392 | M21121=HUMTCSM Human T cell-specific |
| | | protein (RANTES) mRNA |
| RANTES | 11,265 | M21121/FEATURE=/DEFINITION= |
| | | HUMTCSM Human T cell-specific protein |
| | | (RANTES) mRNA |
| RANTES | 1464.2 | M2112=HUMTCSM Human T cell-specific |
| | | protein (RANTES) mRNA |
| SOD3 | 848 | J02947=HUMSODEC Human extracellular- |
| | | superoxide dismutase (SOD3) mRNA |
| sVEGF R (sflt) | 645 | U01134=U01134 Human soluble vascular |
| | | endothelial cell growth factor receptor |
| | | (sflt) mRNA |
| TGF-b | 3,899 | M38449=HUMTGFBA Human transforming |
| | | growth factor-beta mRNA |
| Thymosin | 17,132 | Cluster including M92383:Homo sapiens |
| beta-10 | | thymosin beta-10 gene |
| Thymosin | 19,683 | Cluster including M17733:Human thymosin |
| beta-4 | | beta-4 mRNA |
| Thymosin | 915 | Cluster including AF000989:Homo sapiens |
| beta 4 | | thymosin beta 4 Y isoform (TB4Y) mRNA |
| TNF/LT | 1,392 | M16441=HUMTNFAB Human tumor |
| | | necrosis factor and lymphotoxin genes |
| TNF/LT | 933 | M16441=HUMTNFAB Human tumor |
| | | necrosis factor and lymphotoxin genes |
| TNF-b | 5,506 | Cluster including D12614:Human mRNA for |
| | | lymphotoxin (TNF-beta) |
| TRAIL | 6878.2 | U37518=HSU37518 Human TNF-related |
| | | apoptosis inducing ligand TRAIL mRNA |
| TRAMP | 5,480 | Cluster including X63679:H. sapiens mRNA |
| | | for TRAMP protein |
| VEGF | 2,444 | Cluster including U43368:Human VEGF |
| | | related factor isoform VRF186 precursor |
| | | (VRF) mRNA |

From the foregoing, it can be seen how various objects and features of the invention are met. Those skilled in the art can now appreciate from the foregoing description that the broad teachings of the present invention can be implemented in a variety of forms. Therefore, while this invention has been described in connection with particular embodiments and examples thereof, it will be appreciated that various changes and modification may be made without departing from the invention as claimed.

It is claimed:

1. A composition comprising a mixture of human interferon γ and at least one of human interferon α and human interferon β, in a mole ratio of between 1:1 to 1:1000 interferon γ to interferon α or human interferon β.

2. The composition of claim1, comprising a mixture of human interferon γ and interferon α, in a mole ratio of between 1:10 to 1:100 interferon γ to interferon α.

3. The composition of claim 2, wherein the interferon α includes a mixture of interferon α subtypes, and the mole ratio of human interferon γ and interferon α is calculated on the basis of the combined mole ratio of all of the subtypes present.

4. The composition of claim1, comprising a mixture of human interferon γ and interferon β, in a mole ratio of between 1:10 to 1:100 interferon γ to interferon β.

5. The composition of claim 1, which is formulated in a pharmaceutically injectable solution.

6. The composition of claim 1, which is formulated for dispersion in a particle aerosol, for administration by inhalation.

7. The composition of claim 6, wherein the composition is formulated in a dry powder form.

8. A method for treating a condition in a human subject that is responsive to human interferon α or human interferon β, when administered in a therapeutically effective dose to the subject, comprising
administering to the subject, a dose of human interferon α or human interferon β that is below the dose required for therapeutic efficacy, when administered alone, and
co-administering to the subject, a subclinical dose of human interferon γ.

9. The method of claim 8, for the treatment of viral infection or cancer wherein the interferon administered is interferon α.

10. The method of claim 9, wherein the wherein the interferon α includes a mixture of interferon α subtypes.

11. The method of claim 8, for the treatment of viral infection or cancer wherein the interferon administered is interferon β.

12. The method of claim 8, wherein said interferon γ is co-administered with the interferon α or human interferon β, by administration of a composition containing a mixture of human interferon γ and human interferon α or human interferon β, in a mole ratio of between 1:10 to 1:100 interferon γ to interferon α or human interferon β.

13. The method of claim 12, wherein said composition is formulated in an injectable solution, and is administered by injection.

14. The method of claim 12, wherein said composition is formulated for aerosloization, and is administered by inhalation.

15. A composition for use in human therapy, comprising
a mixture of human interferon α and human interferon β, in a mole ratio of between 1:10 to 10:1, and
a pharmaceutically acceptable carrier.

16. The composition of claim 15, wherein the mixture contains a mole ratio of a mixture of human interferon α to human interferon β between 1:10 and 1:1

17. The composition of claim 15, wherein the mixture contains a mole ratio of a mixture of human interferon α to human interferon β between 1:1 and 1:10.

18. The composition of claim 15, wherein the interferon α includes a mixture of interferon α subtypes, and the mole ratio of human interferon β and interferon α is calculated on the basis of the combined mole ratio of all of the subtypes present.

19. The composition of claim 18, wherein said mixture is produced by culturing interferon-producing cells in culture medium, under conditions of stimulated interferon production, and co-purifying interferon α and human interferon β from the culture medium.

20. The composition of claim 15, which is formulated in a pharmaceutically injectable solution.

21. The composition of claim 15, which is formulated for dispersion in a particle aerosol, for administration by inhalation.

22. The composition of claim 21, wherein the composition is formulated in a dry powder form.

23. A method of treating a viral infection in a human subject, comprising
administering to the subject, a pharmaceutically effective dose of a mixture of human interferon α and human interferon β, in a mole ratio of between 1:10 to 10:1.

24. The method of claim 23, wherein the mixture contains a mole ratio of a mixture of human interferon α human interferon β between 1:10 and 1:1

25. The method of claim 23, wherein the mixture contains a mole ratio of a mixture of human interferon α human interferon β between 1:1 and 1:10.

26. The method of claim 23, wherein the interferon α includes a mixture of interferon α sub types, and the mole ratio of human interferon β and interferon α is calculated on the basis of the combined mole ratio of all of the subtypes present.

27 The method of claim 23, for treating hepatitis C or hepatitis B viruses, wherein said administering is by injection or inhalation.

28. A method of treating cancer in a human subject, comprising
administering to the subject, a pharmaceutically effective dose of a mixture of human interferon α and human interferon β, in a mole ratio of between 1:10 to 10:1.

29. The method of claim 28, wherein the mixture contains a mole ratio of a mixture of human interferon α to human interferon β between 1:10 and 1:1

30. The method of claim 28, wherein the mixture contains a mole ratio of a mixture of human interferon α to human interferon β between 1:1 and 1:10.

31. The method of claim 28, wherein the interferon α includes a mixture of interferon α subtypes, and the mole ratio of human interferon β and interferon α is calculated on the basis of the combined mole ratio of all of the subtypes present.

32 A method of treating multiple sclerosis in a human subject, comprising
administering to the subject, a pharmaceutically effective dose of a mixture of human interferon α and human interferon β, in a mole ratio of between 1:10 to 10:1.
